(19) United States
(12) Patent Application Publication
(10) Pub. No.: US 2023/0158268 A1
(43) Pub. Date: May 25, 2023

(54) CONNECTED OXYGEN THERAPY SYSTEM FOR CHRONIC RESPIRATORY DISEASE MANAGEMENT

(71) Applicant: ResMed Asia Pte. Ltd., Singapore (SG)

(72) Inventors: Rex Dael NAVARRO, Singapore (SG); Yung Tsang MAR, Singapore (SG); Brent James DRAVITZKI, Singapore (SG); Hadley WHITE, Bella Vista, NSW (AU); Kavita CHINOY, Singapore (SG); Eng Lee TAN, Singapore (SG); Palanichamy SARAVANAN, Singapore (SG); Harold Jefferson Mendoza AYSON, Singapore (SG); Khian Boon LIM, Singapore (SG)

(21) Appl. No.: 17/921,910

(22) PCT Filed: Apr. 30, 2021
(86) PCT No.: PCT/IB2021/053650

§ 371 Date: Oct. 27, 2022

Related U.S. Application Data
(60) Provisional application No. 63/018,205, filed on Apr. 30, 2020.

(51) Int. Cl. A61M 16/10; A61M 16/00; A61M 16/20

(52) U.S. Cl. CPC A61M 16/101; A61M 16/026; A61M 16/208; A61M 2230/42; A61M 2230/04; A61M 2230/50

(57) ABSTRACT

A method and system to manage a respiratory condition of a patient. An oxygen concentrator is configured to generate and deliver oxygen enriched air to the patient according to a selected dosage. The oxygen concentrator senses and collects physiological data of the patient and collects operational data during the generation and delivery of oxygen enriched air. The oxygen concentrator adjusts the dosage of oxygen enriched air based on the sensed physiological data. The oxygen concentrator transmits operational data and the physiological data to a health data analysis engine. The health data analysis engine collects the data transmitted by the oxygen concentrator. The health analysis engine detects a triggering event based on the collected data and determines an action to resolve the detected triggering event.

### CROSS-REFERENCE TO RELATED APPLICATIONS

The application claims priority to, and benefit of, U.S. Provisional Patent Application No. 63/018,205, filed Apr. 30, 2020, which is hereby incorporated by reference herein in its entirety.

### TECHNICAL FIELD

The present disclosure relates generally to portable oxygen concentrators (POCs), and more specifically to system that adjusts oxygen output of a POC to specific patient blood oxygenation needs based on cloud based data.

### BACKGROUND

There are many users (patients) that require supplemental oxygen as part of Long Term Oxygen Therapy (LTOT). Currently, the vast majority of users that are receiving LTOT are diagnosed under the general category of Chronic Obstructive Pulmonary Disease (COPD). This general diagnosis includes common diseases such as Chronic Bronchitis, Emphysema, and related pulmonary conditions. Other users may also require supplemental oxygen, for example, obese individuals to maintain elevated activity levels, users with cystic fibrosis or infants with broncho-pulmonary dysplasia.

Doctors may prescribe oxygen concentrators or portable tanks of medical oxygen for these users (patients). Usually a specific continuous oxygen flow rate is prescribed (e.g., 1 litre per minute (LPM), 2 LPM, 3 LPM, etc.). Experts in this field have also recognized that exercise for these users provide long term benefits that slow the progression of the disease, improve quality of life and extend user longevity. Most stationary forms of exercise like treadmills and stationary bicycles, however, are too strenuous for these users. As a result, the need for mobility has long been recognized. Until recently, this mobility has been facilitated by the use of small compressed oxygen tanks or cylinders mounted on a cart with dolly wheels. The disadvantage of these tanks is that they contain a finite amount of oxygen and are heavy, weighing about 50 pounds when mounted.

Oxygen concentrators have been in use for about 50 years to supply oxygen for respiratory therapy. Oxygen concentrators may implement processes such as vacuum swing adsorption (VSA), pressure swing adsorption (PSA), or vacuum pressure swing adsorption (VPSA). For example, oxygen concentrators, e.g., POCs, may work based on depressurization (e.g., vacuum operation) and/or pressurization (e.g., compressor operation) in a swing adsorption process (e.g., Vacuum Swing Adsorption, Pressure Swing Adsorption or Vacuum Pressure Swing Adsorption, each of which are referred to herein as a “swing adsorption process”). Pressure swing adsorption may involve using one or more compressors to increase gas pressure inside one or more canisters that contains particles of a gas separation adsorbent. Such a canister when containing a mass of gas separation adsorbent such as a layer of gas separation adsorbent may serve as a sieve bed. As the pressure increases, certain molecules in the gas may become adsorbed onto the gas separation adsorbent. Removal of a portion of the gas in the canister under the pressurized conditions allows separation of the non-adsorbed molecules from the adsorbed molecules. The adsorbed molecules may then be desorbed by venting the sieve beds. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

Ambient air usually includes approximately 78% nitrogen and 21% oxygen with the balance comprised of argon, carbon dioxide, water vapor and other trace gases. If a gas mixture such as air, for example, is passed under pressure through a canister containing a gas separation adsorbent that attracts nitrogen more strongly than it does oxygen, part or all of the nitrogen will stay in the canister, and the gas coming out of the canister will be enriched in oxygen. When the sieve bed reaches the end of its capacity to adsorb nitrogen, the adsorbed nitrogen may be desorbed by venting. The sieve bed is then ready for another cycle of producing oxygen enriched air. By alternating pressurization cycles of the canisters in a two-canister system, one canister can be separating oxygen while the other canister is being vented (resulting in a near-continuous separation of oxygen from the air). In this manner, oxygen enriched air can be accumulated, such as in a storage container or other pressurizable vessel or conduit coupled to the canisters, for a variety of uses including providing supplemental oxygen to users.

Vacuum swing adsorption (VSA) provides an alternative gas separation technique. VSA typically draws the gas through the separation process of the sieve beds using a vacuum such as a compressor configured to create a vacuum within the sieve beds. Vacuum Pressure Swing Adsorption (VPSA) may be understood to be a hybrid system using a combined vacuum and pressurization technique. For example, a VPSA system may pressurize the sieve beds for the separation process and also apply a vacuum for depressurizing the sieve beds.

Traditional oxygen concentrators have been bulky and heavy making ordinary ambulatory activities with them difficult and impractical. Recently, companies that manufacture large stationary oxygen concentrators began developing portable oxygen concentrators (POCs). The advantage of POCs is that they can produce a theoretically endless supply of oxygen and provide mobility for patients (users). In order to make these devices small for mobility, the various systems necessary for the production of oxygen enriched air are condensed. POCs seek to utilize their produced oxygen as efficiently as possible, in order to minimize weight, size, and power consumption. In some implementations, this may be achieved by delivering the oxygen as series of pulses, each pulse or “bolus” timed to coincide with the onset of inhalation. This therapy mode is known as pulsed oxygen delivery (POD) or demand mode, in contrast with traditional continuous flow delivery more suited to stationary oxygen concentrators. POD mode may be implemented with a conserver, which is essentially an active valve with a sensor to determine the onset of inhalation.

Current POCs are battery powered and output oxygen from air that is filtered by zeolite in a sieve bed. Such sieve beds degrade and at a certain threshold level of oxygen output must be replaced. Current portable oxygen concentrators only pump out oxygen when a user breathes. Current POCs monitor when a patient breathes in by sensing a change in pressure, and then activates the mechanism to inject a certain volume of oxygen during that time.

Ideally, a POC should delivery sufficient supplementary oxygen to keep the blood oxygen saturation (SpO2) level of the user above 90%. However, different users may have different needs or conditions that effect the amount of oxygen required to reach the desired oxygen saturation levels. Further, the need for oxygen may vary on external factors such as the immediate environment as air quality of higher altitudes may result in greater oxygen demand. Another external factor may be physiological changes. For example, if the user is involved in different types of physical activity such as climbing a hill or stairs, their metabolic load will increase, temporarily requiring more oxygen. Thus, current POC designs are inefficient as 90% of the population is either not getting enough oxygen (i.e., ineffective) or getting too much oxygen. The POC is not functioning efficiently if a patient is not receiving sufficient oxygen. If a patient is receiving too much oxygen, the POC suffers from unnecessary draining of batteries and or canisters, resulting in premature exhaustion of such components.

Certain POCs collect operational data for purposes of determining replacement times for different components such as batteries or sieve beds. However, currently, such devices transmit very limited operational data to remote locations via networks such as the cloud. This data is typically supplied using one way communications only, or with very limited bidirectional traffic. Detailed operational data is generally not stored by current devices. No real time chronic disease insights are made available from the limited operational data as such data is generally only used to determine the operation of the POC device and the need for replacement of components such as the battery or the sieve bed.

It would be advantageous for a system to use existing operational data collected by a respiratory therapy device such as a portable oxygen concentrator for chronic respiratory disease management.

### SUMMARY

The present disclosure relates to a connected oxygen therapy system for chronic respiratory disease management.

One disclosed example is a system for managing a respiratory condition of a patient. The system has an oxygen concentrator configured to generate and deliver oxygen enriched air to the patient according to a selected dosage. The oxygen concentrator also senses and collects physiological data of the patient. The oxygen concentrator collects operational data during the generation and delivery of oxygen enriched air. The oxygen concentrator adjusts the dosage of oxygen enriched air based on the sensed physiological data. The oxygen concentrator also transmit operational data and the physiological data. The system includes a health data analysis engine in communication with the oxygen concentrator. The health data analysis engine is configured to collect the data transmitted by the oxygen concentrator; detect a triggering event based on the collected data; and determine a responsive action to resolve the detected triggering event.

A further implementation of the example system is where the oxygen concentrator is a portable oxygen concentrator. The oxygen concentrator includes an air intake, a motorized compressor coupled to the air intake, an oxygen separator to separate oxygen from compressed air from the compressor, and an accumulator coupled to the oxygen separator to store the oxygen enriched air. Another implementation is where the oxygen concentrator has a first data resolution and a second data resolution, where the second data resolution including comparatively more data than the first data resolution. Another implementation is where the operational and physiological data is collected at the second data resolution during a predetermined period of time when the triggering event is detected. Another implementation is where the triggering event is based on long term analysis of the collected data at the first data resolution. Another implementation is where the health data analysis engine is operable to determine a severity of the detected triggering event, and determine the responsive action based on the determined severity. Another implementation is where the operation of the oxygen concentrator is adjusted in response to the triggering event. Another implementation is where the health data analysis engine is operative to request subjective data to be input by the patient to verify the detection of the triggering event. Another implementation is where a health monitoring device coupled to the oxygen concentrator. The health monitoring device includes one or more sensors configured to collect the physiological data. Another implementation is where the one or more sensors of the health monitoring device are selected from one of the group of: an audio sensor, a heart rate sensor, a respiratory sensor, a ECG sensor, a photoplethysmography (PPG) sensor, an infrared sensor, a photoacoustic exhaled carbon dioxide sensor, an activity sensor, a radio frequency sensor, a SONAR sensor, an optical sensor, a Doppler radar motion sensor, a thermometer, an impedance sensor, a piezoelectric sensor, a photoelectric sensor, or a strain gauge sensor. Another implementation is where the health data analysis engine is operable to predict respiratory exacerbations based on the operational data and the physiological data. Another implementation includes a mobile computing device that is operable to receive the data from the oxygen concentrator and transmit the data to the health data analysis engine. Another implementation is where the health data analysis engine is configured to analyze environmental data related to the patient in detecting the triggering event. Another implementation is where the health data analysis engine is configured to analyze demographic data related to the patient in detecting the triggering event. Another implementation is where the responsive action is an adjustment to a treatment of the patient. Another implementation is where the health data analysis engine is operable to determine the results of the treatment for the patient based on the operational and physiological data. Another implementation is where the treatment is medication, and the health data analysis engine is operable to adjust the medication according to the determined results. Another implementation is where the responsive action is a notification to the patient to adjust a treatment. Another implementation is where the responsive action is a notification to a health care professional to adjust a treatment. Another implementation is where the dosage of oxygen enriched air is determined by comparison to a baseline determined from previous physiological data from the patient. Another implementation is where the baseline is based on one or more of: a situation of the patient, a location of the patient, and a time the physiological data is collected. Another implementation is where the triggering event is determined from previous physiological data from a normative patient population related to the patient. Another implementation is where the oxygen concentrator is configured to sense and collect physiological data when oxygen enriched air is not being delivered. Another implementation includes a blood oxygenation sensor coupled to the oxygen concentrator. The physiological data includes blood oxygenation data of the patient measured by the blood oxygenation sensor. Another implementation is where a dead dosage level is identified for the patient where no further increase in blood oxygenation may be achieved.

Another disclosed example is method of managing a respiratory condition of a patient. Oxygen enriched air is generated by an oxygen concentrator. The oxygen enriched air is delivered to the patient according to a selected dosage. Physiological data of the patient is sensed. Operational data is collected during the generation and delivery of oxygen enriched air from the oxygen concentrator. The dosage of oxygen enriched air is adjusted based on the sensed physiological data. The operational data and the physiological data are transmitted to a health data analysis engine in communication with the oxygen concentrator. The data transmitted by the oxygen concentrator is collected. A triggering event based on the collected data is detected. A responsive action is determined to resolve the detected triggering event.

A further implementation of the example system is where the oxygen concentrator is a portable oxygen concentrator. The oxygen concentrator includes an air intake, a motorized compressor coupled to the air intake, an oxygen separator to separate oxygen from compressed air from the compressor, and an accumulator coupled to the oxygen separator to store the oxygen enriched air. Another implementation is where the oxygen concentrator has a first data resolution and a second data resolution, the second data resolution including comparatively more data than the first data resolution. Another implementation is where the operational and physiological data is collected at the second data resolution during a predetermined period of time when the triggering event is detected. Another implementation is where the triggering event is based on long term analysis of the collected data at the first data resolution. Another implementation includes determining a severity of the detected triggering event, and determining the responsive action based on the determined severity. Another implementation includes adjusting the operation of the oxygen concentrator in response to the triggering event. Another implementation includes requesting subjective data to be input by the patient to verify the detection of the triggering event. Another implementation includes collecting physiological data via a health monitoring device coupled to the oxygen concentrator. The health monitoring device includes one or more sensors configured to collect the physiological data. Another implementation is where the one or more sensors of the health monitoring device are selected from one of the group of: an audio sensor, a heart rate sensor, a respiratory sensor, a ECG sensor, a photoplethysmography (PPG) sensor, an infrared sensor, a photoacoustic exhaled carbon dioxide sensor, an activity sensor, a radio frequency sensor, a SONAR sensor, an optical sensor, a Doppler radar motion sensor, a thermometer, an impedance sensor, a piezoelectric sensor, a photoelectric sensor, or a strain gauge sensor. Another implementation includes predicting respiratory exacerbations based on the operational data and the physiological data. Another implementation includes receiving the data from the oxygen concentrator via a mobile computing device. The mobile computing device transmits the data to the health data analysis engine. Another implementation includes analyzing environmental data related to the patient in detecting the triggering event. Another implementation includes analyzing demographic data related to the patient in detecting the triggering event. Another implementation is where the responsive action is an adjustment to a treatment of the patient. Another implementation includes determining the results of the treatment for the patient based on the operational and physiological data. Another implementation is where the treatment is medication, and where the method includes adjusting the medication according to the determined results. Another implementation is where the responsive action is a notification to the patient to adjust a treatment. Another implementation is where the responsive action is a notification to a health care professional to adjust a treatment. Another implementation is where the dosage of oxygen enriched air is determined by comparison to a baseline determined from previous physiological data from the patient. Another implementation is where the baseline is based on one or more of: a situation of the patient, a location of the patient, and a time the physiological data is collected. Another implementation is where the triggering event is determined from previous physiological data from a normative patient population related to the patient. Another implementation is where the oxygen concentrator is configured to sense and collect physiological data when oxygen enriched air is not being delivered. Another implementation is where the physiological data includes blood oxygenation data of the patient measured by a blood oxygenation sensor. Another implementation is where a dead dosage level is identified for the patient where no further increase in blood oxygenation may be achieved. Another implementation is a computer program product comprising instructions which, when executed by a computer, cause the computer to carry out the above method.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1A depicts an oxygen concentrator in accordance with one form of the present technology;

FIG. 1B is a schematic diagram of the components of the oxygen concentrator of FIG. 1A;

FIG. 1C is a side view of the main components of the oxygen concentrator of FIG. 1A;

FIG. 1D is a perspective side view of a compression system of the oxygen concentrator of FIG. 1A;

FIG. 1E is a side view of a compression system that includes a heat exchange conduit;

FIG. 1F is a schematic diagram of example outlet components of the oxygen concentrator of FIG. 1A;

FIG. 1G depicts an outlet conduit for the oxygen concentrator of FIG. 1A;

FIG. 1H depicts an alternate outlet conduit for the oxygen concentrator of FIG. 1A;

FIG. 1I is a perspective view of a disassembled canister system for the oxygen concentrator of FIG. 1A;

FIG. 1J is an end view of the canister system of FIG. 1I;

FIG. 1K is an assembled view of the canister system end depicted in FIG. 1J;

FIG. 1L is a view of an opposing end of the canister system of FIG. 1I to that depicted in FIGS. 1J and 1K;

FIG. 1M is an assembled view of the canister system end depicted in FIG. 1L;

FIG. 1N is a block diagram of a communication arrangement of example devices that may be in communication with the oxygen concentrator of FIG. 1A;

FIG. 1O depicts an example control panel for the oxygen concentrator of FIG. 1A;

FIG. 2 is a block diagram of a connected oxygen therapy system that allows data collection from POCs to determine and respond to health conditions of a patient;

FIG. 3 is a flow diagram of a routine to increase the resolution of collection of data based on a triggering event and provide health data analysis thereof;

FIG. 4 is a flow diagram of a method of determining a baseline dosage for a patient situation according to one implementation of the present technology; and

FIG. 5 is a flow diagram of a method of performing closed-loop therapy based on continuous monitoring of SpO2 levels according to one implementation of the present technology.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that leverages operational data collected by oxygen concentrator devices to allow for analysis of patient health conditions in a cloud-based engine and adjustment of oxygen dosage based on the analysis. The system provides a connected-care service value in the health care (and particularly Integrated Care) market, thereby reducing the burden of care by incorporating sensing technology in oxygen concentrator devices and services, supported by communications technologies. The collected data may be employed by a health data analysis engine to make recommendations to change other aspects of respiratory therapy e.g., medication reminder or lifestyle change.

An example oxygen concentrator device such as a portable oxygen concentrator (POC) may monitor and collect operational data such as the oxygen dosage, and physiological data such as breathing rate and inspiratory time. The example POC may also collect physiological data from additional sensors on a connected body patch or health monitoring device with electrical or optical sensing, such as a smart watch, bracelet, or ring. Data could also be integrated from other therapy devices, such as a smart inhaler, used by the patient.

The present disclosure also relates to a portable oxygen concentrator that provides automatic adjustment of the oxygen dosage delivered to achieve a desired blood oxygenation level in the user. The adjustment is based on data derived from physiological, demographic or environmental monitoring of a user. The disclosure also relates to a system that determines baselines for users as to the necessary oxygen dosage provided by a POC to achieve a desired blood oxygenation level for a particular situation (state or activity). The baselines may be based on the correlation between oxygen provided and blood oxygenation data collected from a population of oxygen concentrator users. Baselines may be separately determined based on personal health data, environmental conditions, or physiological data. Collection of oxygen data from POCs may be used to create a standard for oxygen delivery dosages based on analysis of big data collected from the population of POC users.

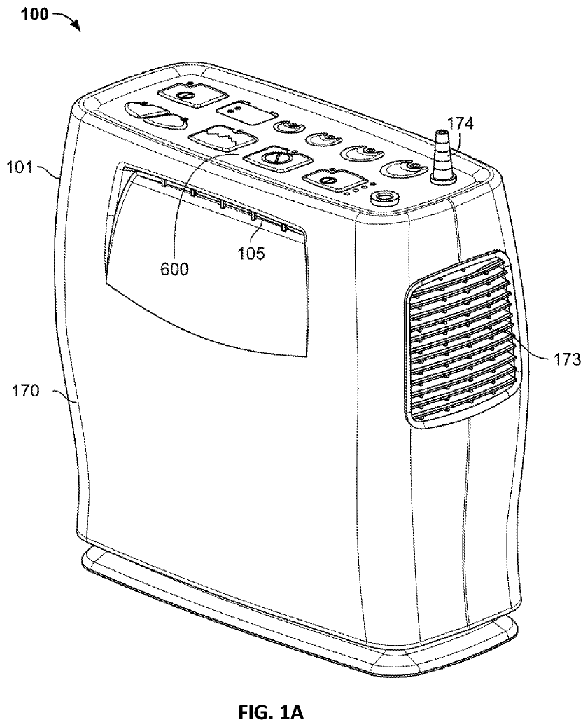

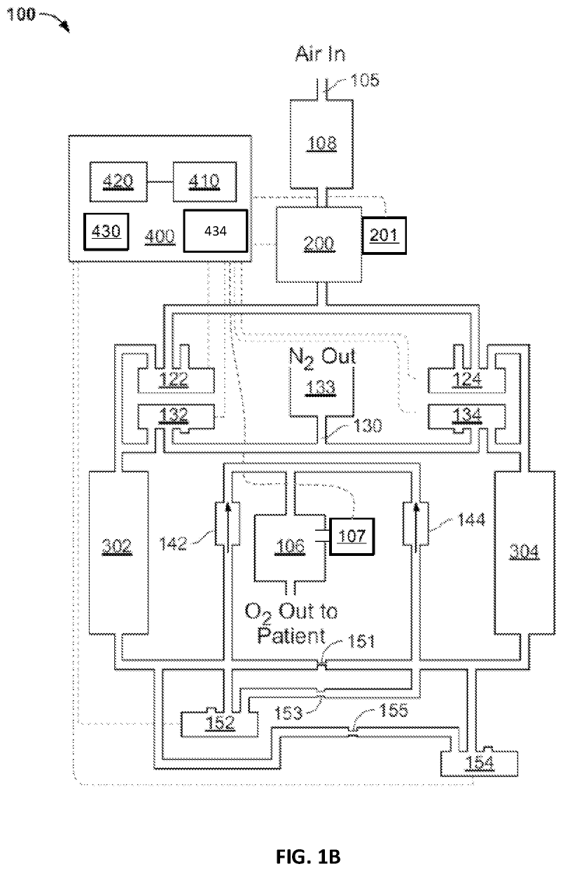

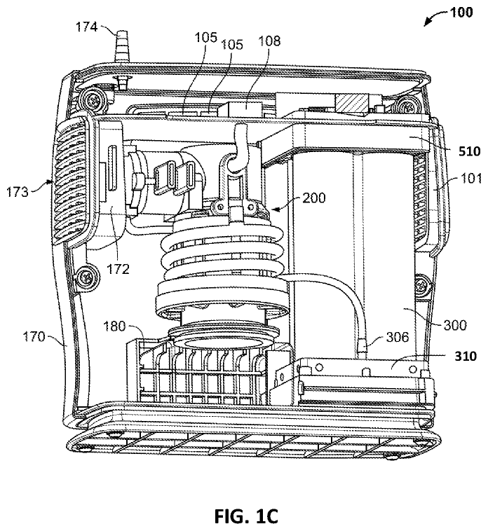

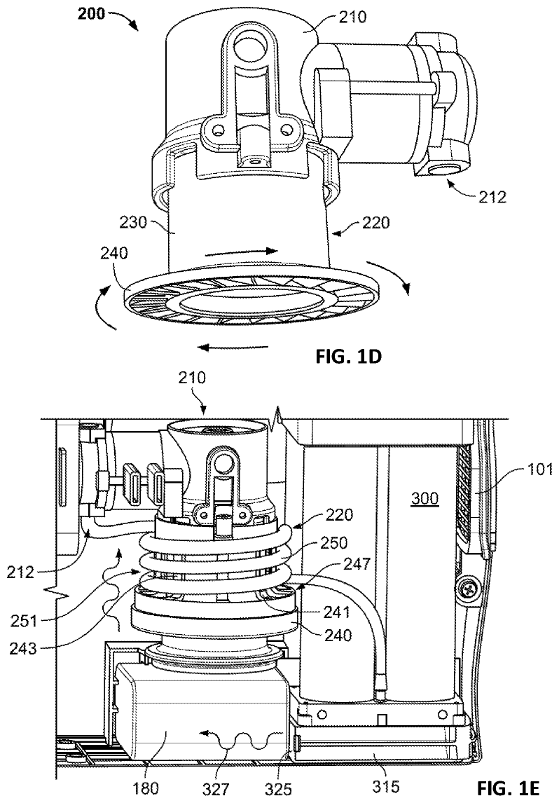

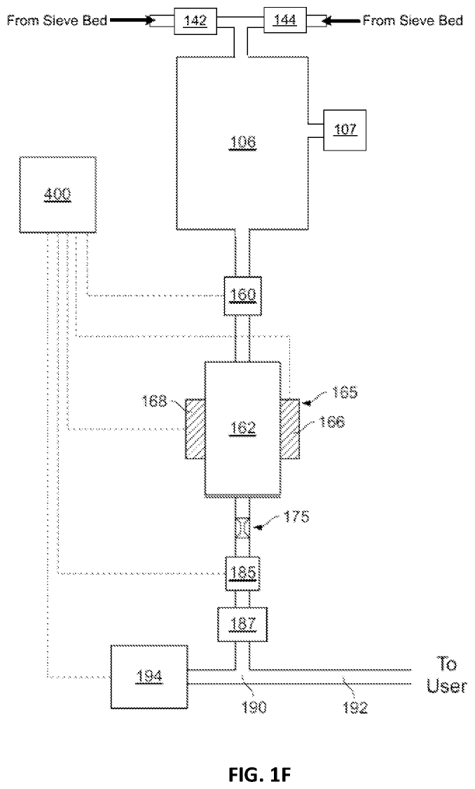

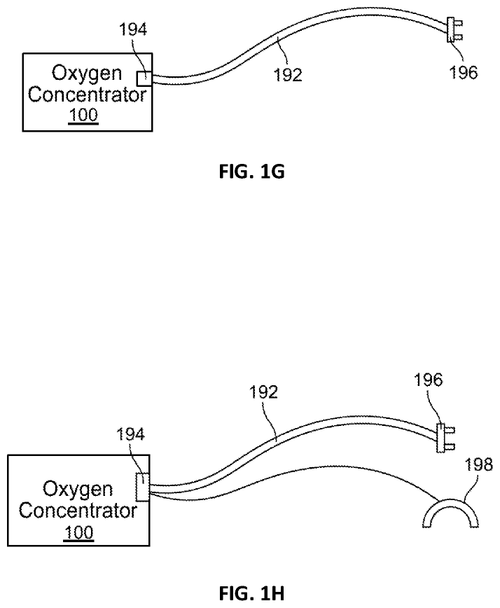

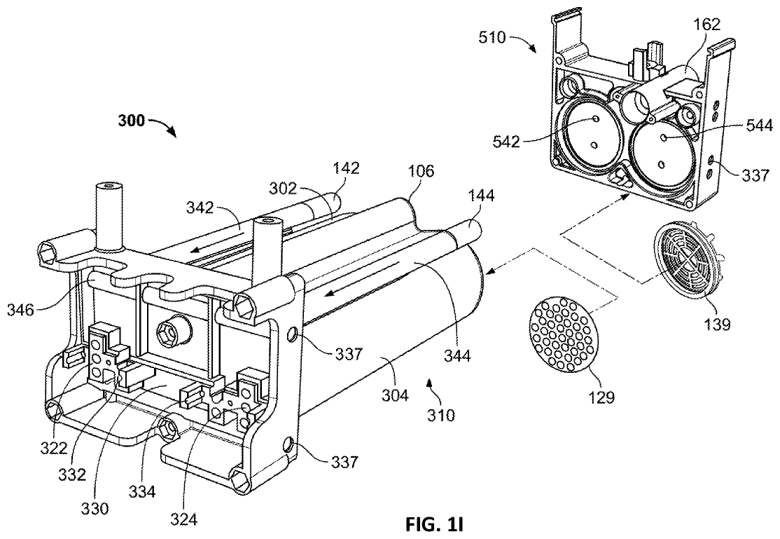

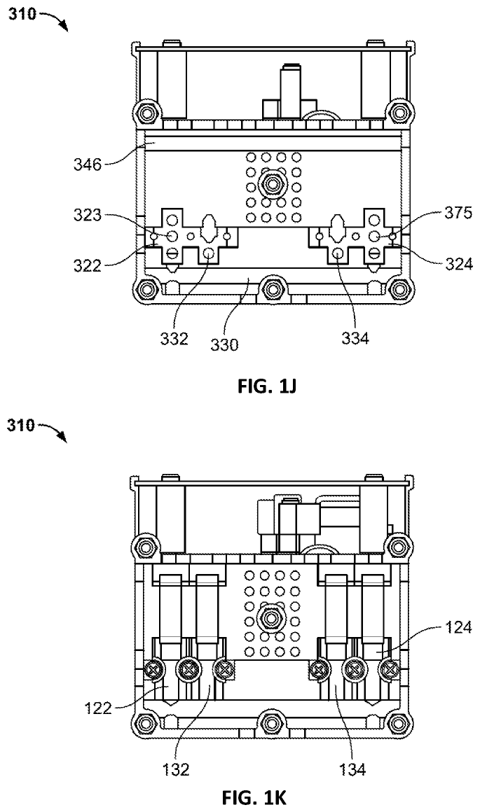

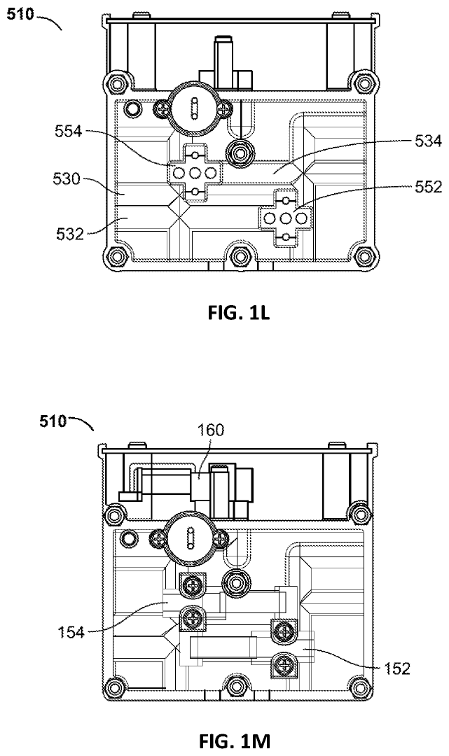

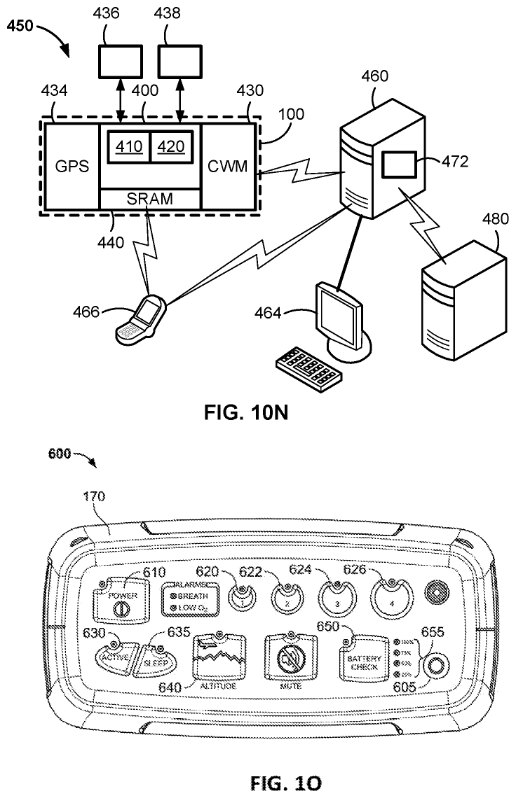

FIGS. 1A-1N illustrate an implementation of an oxygen concentrator 100. As described herein, the oxygen concentrator 100 uses pressure swing adsorption (PSA) processes to produce oxygen enriched air. However, in other embodiments, oxygen concentrator 100 may be modified such that it uses vacuum swing adsorption (VSA) processes or vacuum pressure swing adsorption (VPSA) processes to produce oxygen enriched air. The examples of the present technology may be implemented with any of the following structures and operations.

FIG. 1A depicts an implementation of an outer housing 170 of an oxygen concentrator 100. In some implementations, outer housing 170 may be comprised of a light-weight plastic. The outer housing 170 includes compression system inlets 105, cooling system passive inlet 101 and outlet 173 at each end of the outer housing 170, outlet port 174, and control panel 600. Inlet 101 and outlet 173 allow cooling air to enter the housing, flow through the housing, and exit the interior of housing 170 to aid in cooling of the oxygen concentrator 100. The compression system inlets 105 allow air to enter the compression system. The outlet port 174 is used to attach a conduit to provide oxygen enriched air produced by the oxygen concentrator 100 to a user.

FIG. 1B illustrates a schematic diagram of components of the example oxygen concentrator 100 in FIG. 1A. Oxygen concentrator 100 may concentrate oxygen within an air stream to provide oxygen enriched air to a user. As used herein, “oxygen enriched air” is a gas mixture composed of at least about 50% oxygen, at least about 60% oxygen, at least about 70% oxygen, at least about 80% oxygen, at least about 90% oxygen, at least about 95% oxygen, at least about 98% oxygen, or at least about 99% oxygen. One example of a minimal range is 86-87% oxygen for portable oxygen concentrators.

Oxygen concentrator 100 may be a portable oxygen concentrator. For example, the oxygen concentrator 100 may have a weight and size that allows the oxygen concentrator 100 to be carried by hand and/or in a carrying case. In one implementation, oxygen concentrator 100 has a weight of less than about 20 pounds (9.07 kg), less than about 15 pounds (6.80 kg), less than about 10 pounds (4.54 kg), or less than about 5 pounds (2.27 kg). In an implementation, the oxygen concentrator 100 has a volume of less than about 1000 cubic inches (0.0164 cubic meters), less than about 750 cubic inches (0.0123 cubic meters); less than about 500 cubic inches (0.0082 cubic meters), less than about 250 cubic inches (0.0041 cubic meters), or less than about 200 cubic inches (0.0033 cubic meters).

Oxygen enriched air may be produced from ambient air by pressurizing ambient air in a sieve bed in the form of canisters 302 and 304, which include a gas separation adsorbent. Gas separation adsorbents useful in an oxygen concentrator are capable of separating at least nitrogen from an air stream to produce oxygen enriched air. Examples of gas separation adsorbents include molecular sieves that are capable of separating nitrogen from an air stream. Examples of adsorbents that may be used in an oxygen concentrator include, but are not limited to, zeolites (natural) or synthetic crystalline aluminosilicates that separate nitrogen from an air stream under elevated pressure. Examples of synthetic crystalline aluminosilicates that may be used include, but are not limited to: OXYSIV adsorbents available from UOP LLC, Des Plaines, Ill.; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1B, air may enter the oxygen concentrator 100 through air inlet 105. Air may be drawn into air inlet 105 by compression system 200. Compression system 200 may draw in air from the surroundings of the oxygen concentrator and compress the air, forcing the compressed air into one or both canisters 302 and 304. In an implementation, an inlet muffler 108 may be coupled to air inlet 105 to reduce sound produced by air being pulled into the oxygen concentrator by compression system 200. In an implementation, inlet muffler 108 may be used to reduce moisture and sound. For example, a moisture adsorbent material (such as a polymer water adsorbent material or a zeolite material) may be used to both adsorb moisture, i.e. water, from the incoming air and to reduce the sound of the air passing into the air inlet 105.

Compression system 200 may include one or more compressors configured to compress air. Pressurized air, produced by compression system 200, may be forced into one or both of the canisters 302 and 304. In some implementations, the ambient air may be pressurized in the canisters to a pressure approximately in a range of 13-20 pounds per square inch (psi) (89.6-137.9 kPa) gauge pressure (psig). Other pressures may also be used, depending on the type of gas separation adsorbent disposed in the canisters.

Coupled to each canister 302 and 304 are inlet valves 122 and 124 and outlet valves 132 and 134. As shown in FIG. 1B, the inlet valve 122 is coupled to the canister 302 and the inlet valve 124 is coupled to the canister 304. Outlet valve 132 is coupled to the canister 302 and outlet valve 134 is coupled to canister 304. The inlet valves 122 and 124 are used to control the passage of air from the compression system 200 to the respective canisters. The outlet valves 132 and 134 are used to release gas from the respective canisters 302 and 304 during a venting process. In some implementations, the inlet valves 122 and 124 and the outlet valves 132 and 134 may be silicon plunger solenoid valves. Other types of valves, however, may be used. Plunger valves offer advantages over other kinds of valves by being quiet and having low slippage.

In some implementations, a two-step valve actuation voltage may be generated to control the inlet valves 122 and 124 and the outlet valves 132 and 134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage*Current). This reduction in voltage minimizes heat build up and power consumption to extend run time from the power supply 180 (described below). When the power is cut off to the valve, it closes by spring action. In some implementations, the voltage may be applied as a function of time that is not necessarily a stepped response (e.g., a curved downward voltage between an initial 24 V and a final 7 V).

In an implementation, pressurized air is sent into one of the canisters 302 or 304 while the other canister is being vented. For example, during use, the inlet valve 122 is opened while the inlet valve 124 is closed. Pressurized air from compression system 200 is forced into the canister 302, while being inhibited from entering canister 304 by inlet valve 124. In an implementation, a controller 400 is electrically coupled to valves 122, 124, 132, and 134. The controller 400 includes one or more processors 410 operable to execute program instructions stored in a memory 420. The program instructions configure the controller to perform various predefined methods that are used to operate the oxygen concentrator, such as the methods described in more detail herein. The program instructions may include program instructions for operating the inlet valves 122 and 124 out of phase with each other, i.e., when one of the inlet valves 122 or 124 is opened, the other valve is closed such as when electro-mechanical valve(s) are used. During pressurization of the canister 302, the outlet valve 132 is closed and the outlet valve 134 is opened. Similar to the inlet valves, the outlet valves 132 and 134 are operated out of phase with each other. In some implementations, the voltages and the durations of the voltages used to open the input and output valves may be controlled by controller 400.

Check valves 142 and 144 are coupled to canisters 302 and 304, respectively. Check valves 142 and 144 are one-way valves that are passively operated by the pressure differentials that occur as the canisters are pressurized and vented, or may be active valves. Check valves 142 and 144 are coupled to the canisters to allow oxygen enriched air produced during pressurization of each canister to flow out of the canister, and to inhibit back flow of oxygen enriched air or any other gases into the canister. In this manner, check valves 142 and 144 act as one-way valves allowing oxygen enriched air to exit the respective canisters during pressurization.

The term “check valve,” as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; an umbrella valve; and a lift check valve. Under pressure, nitrogen molecules in the pressurized ambient air are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure reaches a point sufficient to overcome the resistance of the check valve coupled to the canister. In one implementation, the pressure drop of the check valve in the forward direction is less than 1 psi (6.9 kPa). The break pressure in the reverse direction is greater than 100 psi (689.5 kPa). It should be understood, however, that modification of one or more components would alter the operating parameters of these valves. If the forward flow pressure is increased, there is, generally, a reduction in oxygen enriched air production. If the break pressure for reverse flow is reduced or set too low, there is, generally, a reduction in oxygen enriched air pressure.

In an exemplary implementation, the canister 302 is pressurized by compressed air produced in compression system 200 and passed into the canister 302. During pressurization of the canister 302, the inlet valve 122 is open, the outlet valve 132 is closed, the inlet valve 124 is closed and outlet valve 134 is open. The outlet valve 134 is opened when outlet valve 132 is closed to allow substantially simultaneous venting of canister 304 to atmosphere while canister 302 is being pressurized. Canister 302 is pressurized until the pressure in canister is sufficient to open check valve 142. Oxygen enriched air produced in canister 302 exits through check valve 142 and, in one implementation, is collected in an accumulator 106.

After some time, the gas separation adsorbent will become saturated with nitrogen and will be unable to separate significant amounts of nitrogen from incoming air. This point is usually reached after a predetermined time of oxygen enriched air production. In the implementation described above, when the gas separation adsorbent in the canister 302 reaches this saturation point, the inflow of compressed air is stopped and the canister 302 is vented to remove nitrogen. During venting, the inlet valve 122 is closed, and the outlet valve 132 is opened. While the canister 302 is being vented, the canister 304 is pressurized to produce oxygen enriched air in the same manner described above. Pressurization of the canister 304 is achieved by closing the outlet valve 134 and opening the inlet valve 124. The oxygen enriched air exits the canister 304 through the check valve 144.

During venting of the canister 302, the outlet valve 132 is opened allowing pressurized gas (mainly nitrogen) to exit the canister 302 to atmosphere through the concentrator outlet 130. In an implementation, the vented gases may be directed through the muffler 133 to reduce the noise produced by releasing the pressurized gas from the canister. As gas is released from the canister 302, the pressure in the canister 302 drops, allowing the nitrogen to become desorbed from the gas separation adsorbent. The released nitrogen exits the canister 302 through the outlet 130, resetting the canister 302 to a state that allows renewed separation of nitrogen from an air stream. The muffler 133 may include open cell foam (or another material) to muffle the sound of the gas leaving the oxygen concentrator. In some implementations, the combined muffling components/techniques for the input of air and the output of oxygen enriched air may provide for oxygen concentrator operation at a sound level below 50 decibels.

During venting of the canisters 302 and 304, it is advantageous that at least a majority of the nitrogen is removed. In an implementation, at least about 50%, at least about 60%, at least about 70%, at least about 80%, at least about 90%, at least about 95%, at least about 98%, or substantially all of the nitrogen in a canister is removed before the canister is re-used to separate nitrogen from air. In some implementations, nitrogen removal may be assisted using an oxygen enriched air stream that is introduced into the canister from the other canister or stored oxygen enriched air

In an exemplary implementation, a portion of the oxygen enriched air may be transferred from the canister 302 to the canister 304 when the canister 304 is being vented of nitrogen. Transfer of oxygen enriched air from canister 302 to canister 304 during venting of canister 304 helps to desorb nitrogen from the adsorbent by lowering the partial pressure of nitrogen adjacent the adsorbent. The flow of oxygen enriched air also helps to purge desorbed nitrogen (and other gases) from the canister. In an implementation, oxygen enriched air may travel through flow restrictors 151, 153, and 155 between the two canisters 302 and 304. The flow restrictor 151 may be a trickle flow restrictor. The flow restrictor 151, for example, may be a 0.009 D flow restrictor (e.g., the flow restrictor has a radius 0.009″ (0.022 cm) which is less than the diameter of the tube it is inside). Flow restrictors 153 and 155 may be 0.013 D flow restrictors. Other flow restrictor types and sizes are also contemplated and may be used depending on the specific configuration and tubing used to couple the canisters. In some implementations, the flow restrictors may be press fit flow restrictors that restrict air flow by introducing a narrower diameter in their respective tube. In some implementations, the press fit flow restrictors may be made of sapphire, metal or plastic (other materials are also contemplated).

Flow of oxygen enriched air between the canisters may also be controlled by use of a valve 152 and a valve 154. The valves 152 and 154 may be opened for a short duration during the venting process (and may be closed otherwise) to prevent excessive oxygen loss out of the purging canister. Other durations are also contemplated. In an exemplary implementation, the canister 302 is vented and it is desirable to purge the canister 302 by passing a portion of the oxygen enriched air produced in the canister 304 into the canister 302. A portion of oxygen enriched air, upon pressurization of the canister 304, will pass through a flow restrictor 151 into the canister 302 during venting of the canister 302. Additional oxygen enriched air is passed into the canister 302, from the canister 304, through the valve 154 and the flow restrictor 155. The valve 152 may remain closed during the transfer process, or may be opened if additional oxygen enriched air is needed. The selection of appropriate flow restrictors 151 and 155, coupled with controlled opening of the valve 154 allows a controlled amount of oxygen enriched air to be sent from the canister 304 to the canister 302. In an implementation, the controlled amount of oxygen enriched air is an amount sufficient to purge the canister 302 and minimize the loss of oxygen enriched air through venting the valve 132 of the canister 302. While this implementation describes venting of the canister 302, it should be understood that the same process can be used to vent the canister 304 using the flow restrictor 151, the valve 152 and the flow restrictor 153.

The pair of equalization/vent valves 152 and 154 work with the flow restrictors 153 and 155 to optimize the gas flow balance between the two canisters 302 and 304. This may allow for better flow control for venting one of the canisters 302 and 304 with oxygen enriched air from the other of the canisters. It may also provide better flow direction between the two canisters 302 and 304. While the flow valves 152 and 154 may be operated as bi-directional valves, the flow rate through such valves varies depending on the direction of fluid flowing through the valve. For example, oxygen enriched air flowing from the canister 304 toward the canister 302 has a flow rate faster through the valve 152 than the flow rate of oxygen enriched air flowing from the canister 302 toward the canister 304 through the valve 152. If a single valve was to be used, eventually either too much or too little oxygen enriched air would be sent between the canisters and the canisters would, over time, begin to produce different amounts of oxygen enriched air. Use of opposing valves and flow restrictors on parallel air pathways may equalize the flow pattern of the oxygen enriched air between the two canisters. Equalizing the flow may allow for a steady amount of oxygen enriched air to be available to the user over multiple cycles and also may allow a predictable volume of oxygen enriched air to purge the other of the canisters. In some implementations, the air pathway may not have restrictors but may instead have a valve with a built-in resistance or the air pathway itself may have a narrow radius to provide resistance.

At times, the oxygen concentrator 100 may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters 302 and 304 may lead to a negative pressure in the canisters 302 and 304. Valves (e.g., valves 122, 124, 132, and 134) leading to and from the canisters 302 and 304 are dynamically sealed rather than hermetically sealed. Thus, outside air may enter the canisters 302 and 304 after shutdown to accommodate the pressure differential. When outside air enters the canisters 302 and 304, moisture from the outside air may be adsorbed by the gas separation adsorbent. Adsorption of water inside the canisters 302 and 304 may lead to gradual degradation of the gas separation adsorbents, steadily reducing ability of the gas separation adsorbents to produce oxygen enriched air.

In an implementation, outside air may be inhibited from entering the canisters 302 and 304 after the oxygen concentrator 100 is shut down by pressurizing both canisters 302 and 304 prior to shutdown. By storing the canisters 302 and 304 under a positive pressure, the valves may be forced into a hermetically closed position by the internal pressure of the air in the canisters 302 and 304. In an implementation, the pressure in the canisters, 302 and 304 at shutdown, should be at least greater than ambient pressure. As used herein the term “ambient pressure” refers to the pressure of the surroundings in which the oxygen concentrator 200 is located (e.g. the pressure inside a room, outside, in a plane, etc.). In an implementation, the pressure in the canisters 302 and 304, at shut down, is at least greater than standard atmospheric pressure (i.e., greater than 760 mmHg (Torr), 1 atm, 101,325 Pa). In an implementation, the pressure in the canisters 302 and 304, at shutdown, is at least about 1.1 times greater than ambient pressure; is at least about 1.5 times greater than ambient pressure; or is at least about 2 times greater than ambient pressure.

In an implementation, pressurization of the canisters 302 and 304 may be achieved by directing pressurized air into each canister 302 and 304 from the compression system and closing all valves to trap the pressurized air in the canisters. In an exemplary implementation, when a shutdown sequence is initiated, inlet valves 122 and 124 are opened and outlet valves 132 and 134 are closed. Because the inlet valves 122 and 124 are joined together by a common conduit, both canisters 302 and 304 may become pressurized as air and/or oxygen enriched air from one canister may be transferred to the other canister. This situation may occur when the pathway between the compression system and the two inlet valves allows such transfer. Because the oxygen concentrator 100 operates in an alternating pressurize/venting mode, at least one of the canisters 302 and 304 should be in a pressurized state at any given time. In an alternate implementation, the pressure may be increased in each canister 302 and 304 by operation of compression system 200. When the inlet valves 122 and 124 are opened, pressure between the canisters 302 and 304 will equalize, however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

Referring to FIG. 1C, an implementation of an oxygen concentrator 100 is depicted. In this example, the oxygen concentrator 100 includes a compression system 200, a canister system 300, and a power supply 180 disposed within an outer housing 170. Inlets 101 are located in outer housing 170 to allow air from the environment to enter oxygen concentrator 100. The inlets 101 may allow air to flow into the compartment to assist with cooling of the components in the compartment. A power supply 180 provides a source of power for the oxygen concentrator 100. The compression system 200 draws air in through the inlet 105 and a muffler 108. The muffler 108 may reduce noise of air being drawn in by the compression system and also may include a desiccant material to remove moisture, i.e. water, from the incoming air. The oxygen concentrator 100 may further include a fan 172 used to vent air and other gases from the oxygen concentrator via the outlet 173.

In some implementations, the compression system 200 includes one or more compressors. In another implementation, the compression system 200 includes a single compressor, coupled to all of the canisters of the canister system 300. Turning to FIGS. 1D and 1E, a compression system 200 is depicted that includes a compressor 210 and a motor 220. The motor 220 is coupled to the compressor 210 and provides an operating force to the compressor 210 to operate the compression mechanism. For example, the motor 220 may be a motor providing a rotating component that causes cyclical motion of a component of the compressor 210 that compresses air. When the compressor 210 is a piston type compressor, the motor 220 provides an operating force which causes the piston of the compressor 210 to be reciprocated. Reciprocation of the piston causes compressed air to be produced by the compressor 210. The pressure of the compressed air is, in part, estimated by the speed that the compressor is operated at, (e.g., how fast the piston is reciprocated). The motor 220, therefore, may be a variable speed motor that is operable at various speeds to dynamically control the pressure of air produced by the compressor 210.

In one implementation, the compressor 210 includes a single head wobble type compressor having a piston. Other types of compressors may be used such as diaphragm compressors and other types of piston compressors. The motor 220 may be a DC or AC motor and provides the operating power to the compressing component of the compressor 210. The motor 220, in an implementation, may be a brushless DC motor. The motor 220 may be a variable speed motor configured to operate the compressing component of the compressor 210 at variable speeds. The motor 220 may be coupled to a controller 400, as depicted in FIG. 1B, which sends operating signals to the motor to control the operation of the motor. For example, the controller 400 may send signals to the motor 220 to: turn the motor on, turn motor the off, and set the operating speed of motor. Thus, as illustrated in FIG. 1B, the compression system may include a speed sensor 201. The speed sensor may be a motor speed transducer used to determine a rotational velocity of the motor 220 and/or other reciprocating operation of the compression system 200. For example, a motor speed signal from the motor speed transducer may be provided to the controller 400. The speed sensor or motor speed transducer may, for example, be a Hall effect sensor. The controller 400 may operate the compression system via the motor 220 based on the speed signal and/or any other sensor signal of the oxygen concentrator, such as a pressure sensor (e.g., accumulator pressure sensor 107). As illustrated in FIG. 1B, the controller 400 receives sensor signals, such as a speed signal from the speed sensor 201 and accumulator pressure signal from the accumulator pressure sensor 107. With such signal(s), the controller may implement one or more control loops (e.g., feedback control) for operation of the compression system based on sensor signals such as accumulator pressure and/or motor speed as described in more detail herein.

The compression system 200 inherently creates substantial heat. Heat is caused by the consumption of power by the motor 220 and the conversion of power into mechanical motion. The compressor 210 generates heat due to the increased resistance to movement of the compressor components by the air being compressed. Heat is also inherently generated due to adiabatic compression of the air by the compressor 210. Thus, the continual pressurization of air produces heat in the enclosure. Additionally, the power supply 180 may produce heat as power is supplied to the compression system 200. Furthermore, users of the oxygen concentrator may operate the device in unconditioned environments (e.g., outdoors) at potentially higher ambient temperatures than indoors, thus the incoming air will already be in a heated state.

Heat produced inside the oxygen concentrator 100 can be problematic. Lithium ion batteries are generally employed as power supplies for oxygen concentrators due to their long life and light weight. Lithium ion battery packs, however, are dangerous at elevated temperatures and safety controls are employed in the oxygen concentrator 100 to shut down the system if dangerously high power supply temperatures are detected. Additionally, as the internal temperature of the oxygen concentrator 100 increases, the amount of oxygen generated by the concentrator may decrease. This is due, in part, to the decreasing amount of oxygen in a given volume of air at higher temperatures. If the amount of produced oxygen drops below a predetermined amount, the oxygen concentrator 100 may automatically shut down.

Because of the compact nature of oxygen concentrators, dissipation of heat can be difficult. Solutions typically involve the use of one or more fans to create a flow of cooling air through the enclosure. Such solutions, however, require additional power from the power supply 180 and thus shorten the portable usage time of the oxygen concentrator 100. In an implementation, a passive cooling system may be used that takes advantage of the mechanical power produced by the motor 220. Referring to FIGS. 1D and 1E, the motor 220 of the compression system 200 has an external rotating armature 230. Specifically, the armature 230 of the motor 220 (e.g., a DC motor) is wrapped around the stationary field that is driving the armature 230. Since the motor 220 is a large contributor of heat to the overall system it is helpful to transfer heat off the motor and sweep it out of the enclosure. With the external high speed rotation, the relative velocity of the major component of the motor 220 and the air in which it exists is very high. The surface area of the armature is larger if externally mounted than if it is internally mounted. Since the rate of heat exchange is proportional to the surface area and the square of the velocity, using a larger surface area armature mounted externally increases the ability of heat to be dissipated from the motor 220. The gain in cooling efficiency by mounting the armature 230 externally, allows the elimination of one or more cooling fans, thus reducing the weight and power consumption while maintaining the interior of the oxygen concentrator within the appropriate temperature range. Additionally, the rotation of the externally mounted armature 230 creates movement of air proximate to the motor to create additional cooling.

Moreover, an external rotating armature may help the efficiency of the motor, allowing less heat to be generated. A motor having an external armature operates similar to the way a flywheel works in an internal combustion engine. When the motor is driving the compressor, the resistance to rotation is low at low pressures. When the pressure of the compressed air is higher, the resistance to rotation of the motor is higher. As a result, the motor does not maintain consistent ideal rotational stability, but instead surges and slows down depending on the pressure demands of the compressor. This tendency of the motor to surge and then slow down is inefficient and therefore generates heat. Use of an external armature adds greater angular momentum to the motor which helps to compensate for the variable resistance experienced by the motor. Since the motor does not have to work as hard, the heat produced by the motor may be reduced.

In an implementation, cooling efficiency may be further increased by coupling an air transfer device 240 to the external rotating armature 230. In an implementation, the air transfer device 240 is coupled to the external armature 230 such that rotation of the external armature 230 causes the air transfer device 240 to create an air flow that passes over at least a portion of the motor. In an implementation, the air transfer device 240 includes one or more fan blades coupled to the external armature 230. In an implementation, a plurality of fan blades may be arranged in an annular ring such that the air transfer device 240 acts as an impeller that is rotated by movement of the external rotating armature 230. As depicted in FIGS. 1D and 1E, the air transfer device 240 may be mounted to an outer surface of the external armature 230, in alignment with the motor 220. The mounting of the air transfer device 240 to the armature 230 allows air flow to be directed toward the main portion of the external rotating armature 230, providing a cooling effect during use. In an implementation, the air transfer device 240 directs air flow such that a majority of the external rotating armature 230 is in the air flow path.

Further, referring to FIGS. 1D and 1E, air pressurized by the compressor 210 exits the compressor 210 at the compressor outlet 212. A compressor outlet conduit 250 is coupled to the compressor outlet 212 to transfer the compressed air to the canister system 300. As noted previously, compression of air causes an increase in the temperature of the air. This increase in temperature can be detrimental to the efficiency of the oxygen concentrator. In order to reduce the temperature of the pressurized air, the compressor outlet conduit 250 is placed in the air flow path produced by the air transfer device 240. At least a portion of the compressor outlet conduit 250 may be positioned proximate to the motor 220. Thus, air flow, created by air transfer device, may contact both the motor 220 and the compressor outlet conduit 250. In one implementation, a majority of the compressor outlet conduit 250 is positioned proximate to the motor 220. In an implementation, the compressor outlet conduit 250 is coiled around the motor 220, as depicted in FIG. 1E.

In an implementation, the compressor outlet conduit 250 is composed of a heat exchange metal. Heat exchange metals include, but are not limited to, aluminum, carbon steel, stainless steel, titanium, copper, copper-nickel alloys or other alloys formed from combinations of these metals. Thus, the compressor outlet conduit 250 can act as a heat exchanger to remove heat that is inherently caused by compression of the air. By removing heat from the compressed air, the number of molecules in a given volume at a given pressure is increased. As a result, the amount of oxygen enriched air that can be generated by each canister during each pressure swing cycle may be increased.

The heat dissipation mechanisms described herein are either passive or make use of elements required for the oxygen concentrator 100. Thus, for example, dissipation of heat may be increased without using systems that require additional power. By not requiring additional power, the run-time of the battery packs may be increased and the size and weight of the oxygen concentrator may be minimized. Likewise, use of an additional box fan or cooling unit may be eliminated. Eliminating such additional features reduces the weight and power consumption of the oxygen concentrator.

As discussed above, adiabatic compression of air causes the air temperature to increase. During venting of a canister in the canister system 300, the pressure of the gas being released from the canisters decreases. The adiabatic decompression of the gas in the canister causes the temperature of the gas to drop as it is vented. In an implementation, the cooled vented gases 327 from the canister system 300 are directed toward the power supply 180 and toward the compression system 200. In an implementation, a base 315 of canister system 300 receives the vented gases 327 from the canisters. The vented gases 327 are directed through the base 315 toward the outlet 325 of the base and toward the power supply 180. The vented gases, as noted, are cooled due to decompression of the gases and therefore passively provide cooling to the power supply. When the compression system is operated, the air transfer device will gather the cooled vented gases and direct the gases toward the motor 220 of the compression system 200. The fan 172 may also assist in directing the vented gas across the compression system 200 and out of the housing 170. In this manner, additional cooling may be obtained without requiring any further power requirements from the battery.

The oxygen concentrator system 100 may include at least two canisters, each canister including a gas separation adsorbent. The canisters of the oxygen concentrator system 100 may be formed from a molded housing. In an implementation, the prior art canister system 300 (aka sieve bed) includes two housing components 310 and 510, as depicted in FIG. 1I. In various implementations, the housing components 310 and 510 of the oxygen concentrator 100 may form a two-part molded plastic frame that defines the two canisters 302 and 304 and the accumulator 106.

The housing components 310 and 510 may be formed separately and then coupled together. In some implementations, the housing components 310 and 510 may be injection molded or compression molded. The housing components 310 and 510 may be made from a thermoplastic polymer such as polycarbonate, methylene carbide, polystyrene, acrylonitrile butadiene styrene (ABS), polypropylene, polyethylene, or polyvinyl chloride. In another implementation, the housing components 310 and 510 may be made of a thermoset plastic or metal (such as stainless steel or a lightweight aluminum alloy). Lightweight materials may be used to reduce the weight of the oxygen concentrator 100. In some implementations, the two housings 310 and 510 may be fastened together using screws or bolts. Alternatively, the housing components 310 and 510 may be solvent welded together.

As shown, the valve seats 322, 324, 332, and 334 and air pathways 330 and 346 may be integrated into the housing component 310 to reduce the number of sealed connections needed throughout the air flow of the oxygen concentrator 100.

Air pathways/tubing between different sections in the housing components 310 and 510 may take the form of molded conduits. Conduits in the form of molded channels for air pathways may occupy multiple planes in the housing components 310 and 510. For example, the molded air conduits may be formed at different depths and at different x, y, z positions in the housing components 310 and 510. In some implementations, a majority or substantially all of the conduits may be integrated into the housing components 310 and 510 to reduce potential leak points.

In some implementations, prior to coupling the housing components 310 and 510 together, O-rings may be placed between various points of the housing components 310 and 510 to ensure that the housing components are properly sealed. In some implementations, components may be integrated and/or coupled separately to the housing components 310 and 510. For example, tubing, flow restrictors (e.g., press fit flow restrictors), oxygen sensors, gas separation adsorbents, check valves, plugs, processors, power supplies, etc. may be coupled to the housing components 310 and 510 before and/or after the housing components are coupled together.

In some implementations, apertures 337 leading to the exterior of the housing components 310 and 510 may be used to insert devices such as flow restrictors. Apertures may also be used for increased moldability. One or more of the apertures may be plugged after molding (e.g., with a plastic plug). In some implementations, flow restrictors may be inserted into passages prior to inserting plugs to seal the passages. Press fit flow restrictors may have diameters that may allow a friction fit between the press fit flow restrictors and their respective apertures. In some implementations, an adhesive may be added to the exterior of the press fit flow restrictors to hold the press fit flow restrictors in place once inserted. In some implementations, the plugs may have a friction fit with their respective tubes (or may have an adhesive applied to their outer surface). The press fit flow restrictors and/or other components may be inserted and pressed into their respective apertures using a narrow tip tool or rod (e.g., with a diameter less than the diameter of the respective aperture). In some implementations, the press fit flow restrictors may be inserted into their respective tubes until they abut a feature in the tube to halt their insertion. For example, the feature may include a reduction in radius. Other features are also contemplated (e.g., a bump in the side of the tubing, threads, etc.). In some implementations, press fit flow restrictors may be molded into the housing components (e.g., as narrow tube segments).

In some implementations, a spring baffle 139 may be placed into respective canister receiving portions of the housing components 310 and 510 with the spring side of the baffle 139 facing the exit of the canister. The spring baffle 139 may apply force to gas separation adsorbent in the canister while also assisting in preventing gas separation adsorbent from entering the exit apertures. Use of the spring baffle 139 may keep the gas separation adsorbent compact while also allowing for expansion (e.g., thermal expansion). Keeping the gas separation adsorbent compact may prevent the gas separation adsorbent from breaking during movement of the oxygen concentrator system 100.

In some implementations, a filter 129 may be placed into respective canister receiving portions of the housing components 310 and 510 facing the inlet of the respective canisters. The filter 129 removes particles from the feed gas stream entering the canisters.

In some implementations, pressurized air from the compression system 200 may enter an air inlet 306. The air inlet 306 is coupled to an inlet conduit 330. Air enters the housing component 310 through the inlet 306, travels through the conduit 330, and then to the valve seats 322 and 324. FIG. 1J and FIG. 1K depict an end view of the housing 310. FIG. 1J depicts an end view of the housing 310 prior to fitting valves to the housing 310. FIG. 1K depicts an end view of the housing 310 with the valves fitted to the housing 310. The valve seats 322 and 324 are configured to receive the inlet valves 122 and 124 respectively. The inlet valve 122 is coupled to the canister 302 and the inlet valve 124 is coupled to the canister 304. The housing 310 also includes the valve seats 332 and 334 configured to receive the outlet valves 132 and 134 respectively. The outlet valve 132 is coupled to the canister 302 and the outlet valve 134 is coupled to the canister 304. The inlet valves 122 and 124 are used to control the passage of air from the conduit 330 to the respective canisters.

In an implementation, pressurized air is sent into one of canisters 302 or 304 while the other canister is being vented. For example, during use, the inlet valve 122 is opened while the inlet valve 124 is closed. Pressurized air from the compression system 200 is forced into the canister 302, while being inhibited from entering the canister 304 by the inlet valve 124. During pressurization of the canister 302, the outlet valve 132 is closed and the outlet valve 134 is opened. Similar to the inlet valves 122 and 124, the outlet valves 132 and 134 are operated out of phase with each other. The valve seat 322 includes an opening 323 that passes through the housing 310 into the canister 302. Similarly, the valve seat 324 includes an opening 375 that passes through the housing 310 into the canister 302. Air from the conduit 330 passes through the openings 323 or 375 if the respective valves 322 and 324 are open, and enters a canister.

Check valves 142 and 144 (See FIG. 1I) are coupled to the canisters 302 and 304, respectively. The check valves 142 and 144 are one way valves that are passively operated by the pressure differentials that occur as the canisters are pressurized and vented. Oxygen enriched air produced in the canisters 302 and 304 passes from the canisters into the openings 542 and 544 of the housing component 510. A passage (not shown) links the openings 542 and 544 to the conduits 342 and 344, respectively. Oxygen enriched air produced in the canister 302 passes from the canister 302 though the opening 542 and into the conduit 342 when the pressure in the canister 302 is sufficient to open the check valve 142. When the check valve 142 is open, oxygen enriched air flows through the conduit 342 toward the end of the housing 310. Similarly, oxygen enriched air produced in the canister 304 passes from the canister 304 through the opening 544 and into the conduit 344 when the pressure in the canister 304 is sufficient to open the check valve 144. When the check valve 144 is open, oxygen enriched air flows through the conduit 344 toward the end of the housing 310.

Oxygen enriched air from either canister 302 or 304 travels through the conduit 342 or 344 and enters the conduit 346 formed in the housing 310. The conduit 346 includes openings that couple the conduit to the conduit 342, the conduit 344 and the accumulator 106. Thus, oxygen enriched air, produced in the canister 302 or 304, travels to conduit 346 and passes into the accumulator 106. As illustrated in FIG. 1B, gas pressure within the accumulator 106 may be measured by a sensor, such as with an accumulator pressure sensor 107. (See also FIG. 1F.) Thus, the accumulator pressure sensor provides a signal representing the pressure of the accumulated oxygen enriched air. An example of a suitable pressure transducer is a sensor from the HONEYWELL ASDX series. An alternative suitable pressure transducer is a sensor from the NPA Series from GENERAL ELECTRIC. In some versions, the pressure sensor may alternatively measure pressure of the gas outside of the accumulator 106, such as in an output path between the accumulator 106 and a valve (e.g., supply valve 160) that controls the release of the oxygen enriched air for delivery to a user in a bolus.

After some time, the gas separation adsorbent will become saturated with nitrogen and will be unable to separate significant amounts of nitrogen from incoming air. When the gas separation adsorbent in a canister reaches this saturation point, the inflow of compressed air is stopped and the canister is vented to remove nitrogen. The canister 302 is vented by closing the inlet valve 122 and opening the outlet valve 132. The outlet valve 132 releases the vented gas from the canister 302 into the volume defined by the end of the housing 310. Foam material may cover the end of the housing 310 to reduce the sound made by release of gases from the canisters. Similarly, the canister 304 is vented by closing the inlet valve 124 and opening outlet the valve 134. The outlet valve 134 releases the vented gas from the canister 304 into the volume defined by the end of the housing 310.

While the canister 302 is being vented, the canister 304 is pressurized to produce oxygen enriched air in the same manner described above. Pressurization of the canister 304 is achieved by closing the outlet valve 134 and opening the inlet valve 124. The oxygen enriched air exits the canister 304 through the check valve 144.

In an exemplary implementation, a portion of the oxygen enriched air may be transferred from the canister 302 to the canister 304 when the canister 304 is being vented of nitrogen. Transfer of oxygen enriched air from the canister 302 to the canister 304, during venting of the canister 304, helps to desorb nitrogen from the adsorbent by lowering the partial pressure of nitrogen adjacent the adsorbent. The flow of oxygen enriched air also helps to purge desorbed nitrogen (and other gases) from the canister 304. Flow of oxygen enriched air between the canisters 302 and 304 is controlled using flow restrictors and valves, as depicted in FIG. 1B. Three conduits are formed in the housing component 510 for use in transferring oxygen enriched air between the canisters 302 and 304. As shown in FIG. 1L, the conduit 530 couples the canister 302 to the canister 304. The flow restrictor 151 (not shown) is disposed in the conduit 530, between the canister 302 and the canister 304 to restrict flow of oxygen enriched air during use. The conduit 532 also couples the canister 302 to the canister 304. The conduit 532 is coupled to the valve seat 552 which receives the valve 152, as shown in FIG. 1M. The flow restrictor 153 (not shown) is disposed in the conduit 532, between the canister 302 and the canister 304. The conduit 534 also couples the canister 302 to the canister 304. The conduit 534 is coupled to the valve seat 554 which receives the valve 154, as shown in FIG. 1M. The flow restrictor 155 (not shown) is disposed in the conduit 534, between the canister 302 and the canister 304. The pair of equalization/vent valves 152/154 work with the flow restrictors 153 and 155 to optimize the air flow balance between the two canisters 302 and 304.

Oxygen enriched air in the accumulator 106 passes through the supply valve 160 into the expansion chamber 162 which is formed in the housing component 510. An opening (not shown) in the housing component 510 couples accumulator 106 to the supply valve 160. In an implementation, the expansion chamber 162 may include one or more devices configured to estimate an oxygen concentration of gas passing through the chamber.

An outlet system, coupled to one or more of the canisters, includes one or more conduits for providing oxygen enriched air to a user. In an implementation, oxygen enriched air produced in either of the canisters 302 and 304 is collected in the accumulator 106 through the check valves 142 and 144, respectively, as depicted schematically in FIG. 1B. The oxygen enriched air leaving the canisters 302 and 304 may be collected in the oxygen accumulator 106 prior to being provided to a user. In some implementations, a tube may be coupled to the accumulator 106 to provide the oxygen enriched air to the user. Oxygen enriched air may be provided to the user through an airway delivery device (e.g., a patient interface) that transfers the oxygen enriched air to the user's mouth and/or nose. In an implementation, an outlet may include a tube that directs the oxygen toward a user's nose and/or mouth that may not be directly coupled to the user's nose.

Turning to FIG. 1F, a schematic diagram of an implementation of an outlet system for an oxygen concentrator is shown. A supply valve 160 may be coupled to an outlet tube to control the release of the oxygen enriched air from the accumulator 106 to the user. In an implementation, supply valve 160 is an electromagnetically actuated plunger valve. The supply valve 160 is actuated by the controller 400 to control the delivery of oxygen enriched air to a user. Actuation of supply valve 160 is not timed or synchronized to the pressure swing adsorption process. Instead, actuation is synchronized to the user's breathing as described below. In some implementations, the supply valve 160 may have continuously-valued actuation to establish a clinically effective amplitude profile for providing oxygen enriched air.

Oxygen enriched air in the accumulator 106 passes through the supply valve 160 into the expansion chamber 162 as depicted in FIG. 1F. In an implementation, the expansion chamber 162 may include one or more devices configured to estimate an oxygen concentration of gas passing through the expansion chamber 162. Oxygen enriched air in the expansion chamber 162 builds briefly, through release of gas from the accumulator 106 by the supply valve 160, and then is bled through a small orifice flow restrictor 175 to a flow rate sensor 185 and then to a particulate filter 187. The flow restrictor 175 may be a 0.025 D flow restrictor. Other flow restrictor types and sizes may be used. In some implementations, the diameter of the air pathway in the housing may be restricted to create restricted gas flow. The flow rate sensor 185 may be any sensor configured to generate a signal representing the rate of gas flowing through the conduit. A particulate filter 187 may be used to filter bacteria, dust, granule particles, etc., prior to delivery of the oxygen enriched air to the user. The oxygen enriched air passes through the filter 187 to the connector 190 which sends the oxygen enriched air to the user via the delivery conduit 192 and to the pressure sensor 194.

The fluid dynamics of the outlet pathway, coupled with the programmed actuations of the supply valve 160, may result in a bolus of oxygen being provided at the correct time and with an amplitude profile that assures rapid delivery into the user's lungs without excessive waste.

The expansion chamber 162 may include one or more oxygen sensors adapted to determine an oxygen concentration of gas passing through the chamber. In an implementation, the oxygen concentration of gas passing through the expansion chamber 162 is estimated using an oxygen sensor 165. An oxygen sensor is a device configured to measure oxygen concentration in a gas. Examples of oxygen sensors include, but are not limited to, ultrasonic oxygen sensors, electrical oxygen sensors, chemical oxygen sensors, and optical oxygen sensors. In one implementation, the oxygen sensor 165 is an ultrasonic oxygen sensor that includes an ultrasonic emitter 166 and an ultrasonic receiver 168. In some implementations, the ultrasonic emitter 166 may include multiple ultrasonic emitters and the ultrasonic receiver 168 may include multiple ultrasonic receivers. In implementations having multiple emitters/receivers, the multiple ultrasonic emitters and multiple ultrasonic receivers may be axially aligned (e.g., across the gas flow path which may be perpendicular to the axial alignment).

In use, the ultrasonic sound wave from emitter 166 may be directed through oxygen enriched air disposed in the chamber 162 to the receiver 168. The ultrasonic oxygen sensor 165 may be configured to detect the speed of sound through the oxygen enriched air to determine the composition of the oxygen enriched air. The speed of sound is different in nitrogen and oxygen, and in a mixture of the two gases, the speed of sound through the mixture may be an intermediate value proportional to the relative amounts of each gas in the mixture. In use, the sound at the receiver 168 is slightly out of phase with the sound sent from the emitter 166. This phase shift is due to the relatively slow velocity of sound through a gas medium as compared with the relatively fast speed of the electronic pulse through wire. The phase shift, then, is proportional to the distance between the emitter 166 and the receiver 168 and inversely proportional to the speed of sound through the expansion chamber 162. The density of the gas in the chamber 162 affects the speed of sound through the expansion chamber 162 and the density is proportional to the ratio of oxygen to nitrogen in the expansion chamber 162. Therefore, the phase shift can be used to measure the concentration of oxygen in the expansion chamber 162. In this manner the relative concentration of oxygen in the accumulator 106 may be estimated as a function of one or more properties of a detected sound wave traveling through the accumulator 106.

In some implementations, multiple emitters 166 and receivers 168 may be used. The readings from the emitters 166 and receivers 168 may be averaged to reduce errors that may be inherent in turbulent flow systems. In some implementations, the presence of other gases may also be detected by measuring the transit time and comparing the measured transit time to predetermined transit times for other gases and/or mixtures of gases.

The sensitivity of the ultrasonic sensor system may be increased by increasing the distance between the emitter 166 and the receiver 168, for example to allow several sound wave cycles to occur between the emitter 166 and the receiver 168. In some implementations, if at least two sound cycles are present, the influence of structural changes of the transducer may be reduced by measuring the phase shift relative to a fixed reference at two points in time. If the earlier phase shift is subtracted from the later phase shift, the shift caused by thermal expansion of the expansion chamber 162 may be reduced or cancelled. The shift caused by a change of the distance between the emitter 166 and the receiver 168 may be approximately the same at the measuring intervals, whereas a change owing to a change in oxygen concentration may be cumulative. In some implementations, the shift measured at a later time may be multiplied by the number of intervening cycles and compared to the shift between two adjacent cycles. Further details regarding sensing of oxygen in the expansion chamber may be found, for example, in U.S. patent application Ser. No. 12/163,549, entitled “Oxygen Concentrator Apparatus and Method”, which published as U.S. Publication No. 2009/0065007 A1 on Mar. 12, 2009 and is incorporated herein by reference.

The flow rate sensor 185 may be used to determine the flow rate of gas flowing through the outlet system. Flow rate sensors that may be used include, but are not limited to: diaphragm/bellows flow meters; rotary flow meters (e.g. Hall effect flow meters); turbine flow meters; orifice flow meters; and ultrasonic flow meters. The flow rate sensor 185 may be coupled to the controller 400. The rate of gas flowing through the outlet system may be an indication of the breathing volume of the user. Changes in the flow rate of gas flowing through the outlet system may also be used to determine a breathing rate of the user. The controller 400 may generate a control signal or trigger signal to control actuation of the supply valve 160. Such control of actuation of the supply valve may be based on the breathing rate and/or breathing volume of the user, as estimated by the flow rate sensor 185.

In some implementations, the ultrasonic sensor 165 and, for example, the flow rate sensor 185 may provide a measurement of an actual amount of oxygen being provided. For example, the flow rate sensor 185 may measure a volume of gas (based on flow rate) provided and the ultrasonic sensor 165 may provide the concentration of oxygen of the gas provided. These two measurements together may be used by the controller 400 to determine an approximation of the actual amount of oxygen provided to the user.

Oxygen enriched air passes through the flow rate sensor 185 to the filter 187. The filter 187 removes bacteria, dust, granule particles, etc. prior to providing the oxygen enriched air to the user. The filtered oxygen enriched air passes through the filter 187 to the connector 190. The connector 190 may be a “Y” connector coupling the outlet of the filter 187 to the pressure sensor 194 and the delivery conduit 192. The pressure sensor 194 may be used to monitor the pressure of the gas passing through the conduit 192 to the user. In some implementations, the pressure sensor 194 is configured to generate a signal that is proportional to the amount of positive or negative pressure applied to a sensing surface. Changes in pressure, sensed by the pressure sensor 194, may be used to determine a breathing rate of a user, as well as the onset of inhalation (also referred to as the trigger instant) as described below. The controller 400 may control actuation of the supply valve 160 based on the breathing rate and/or onset of inhalation of the user. In an implementation, the controller 400 may control actuation of the supply valve 160 based on information provided by either or both of the flow rate sensor 185 and the pressure sensor 194. As will be explained below, the controller 400 adjusts the volume of each bolus by controlling the time the supply valve 160 is actuated. The controller 400 may calibrate the time of actuation and bolus volume by reading data from the ultrasonic sensor 165 and the flow rate sensor 185 so as to provide a measurement of the actual volume (dosage) of oxygen being provided.

Oxygen enriched air may be provided to a user through the delivery conduit 192. In an implementation, the delivery conduit 192 may be a silicone tube. The delivery conduit 192 may be coupled to a user using an airway delivery device 196, as depicted in FIGS. 1G and 1H. The airway delivery device 196 may be any device capable of providing the oxygen enriched air to nasal cavities or oral cavities. Examples of airway delivery devices include, but are not limited to: nasal masks, nasal pillows, nasal prongs, nasal cannulas, and mouthpieces. A nasal cannula airway delivery device 196 is depicted in FIG. 1G. The nasal cannula airway delivery device 196 is positioned proximate to a user's airway (e.g., proximate to the user's mouth and or nose) to allow delivery of the oxygen enriched air to the user while allowing the user to breathe air from the surroundings.

In an alternate implementation, a mouthpiece may be used to provide oxygen enriched air to the user. As shown in FIG. 1H, a mouthpiece 198 may be coupled to the oxygen concentrator 100. The mouthpiece 198 may be the only device used to provide oxygen enriched air to the user, or a mouthpiece may be used in combination with a nasal delivery device 196 (e.g., a nasal cannula). As depicted in FIG. 1H, oxygen enriched air may be provided to a user through both a nasal airway delivery device 196 and a mouthpiece 198.

The mouthpiece 198 is removably positionable in a user's mouth. In one implementation, the mouthpiece 198 is removably couplable to one or more teeth in a user's mouth. During use, oxygen enriched air is directed into the user's mouth via the mouthpiece. The mouthpiece 198 may be a night guard mouthpiece which is molded to conform to the user's teeth. Alternatively, mouthpiece may be a mandibular repositioning device. In an implementation, at least a majority of the mouthpiece is positioned in a user's mouth during use.

During use, oxygen enriched air may be directed to the mouthpiece 198 when a change in pressure is detected proximate to the mouthpiece. In one implementation, the mouthpiece 198 may be coupled to a pressure sensor 194. When a user inhales air through the user's mouth, the pressure sensor 194 may detect a drop in pressure proximate to the mouthpiece. The controller 400 of the oxygen concentrator 100 may control release of a bolus of oxygen enriched air to the user at the onset of inhalation.

During typical breathing of an individual, inhalation may occur through the nose, through the mouth or through both the nose and the mouth. Furthermore, breathing may change from one passageway to another depending on a variety of factors. For example, during more active activities, a user may switch from breathing through their nose to breathing through their mouth, or breathing through their mouth and nose. A system that relies on a single mode of delivery (either nasal or oral), may not function properly if breathing through the monitored pathway is stopped. For example, if a nasal cannula is used to provide oxygen enriched air to the user, an inhalation sensor (e.g., a pressure sensor or flow rate sensor) is coupled to the nasal cannula to determine the onset of inhalation. If the user stops breathing through their nose, and switches to breathing through their mouth, the oxygen concentrator 100 may not know when to provide the oxygen enriched air since there is no feedback from the nasal cannula. Under such circumstances, the oxygen concentrator 100 may increase the flow rate and/or increase the frequency of providing oxygen enriched air until the inhalation sensor detects an inhalation by the user. If the user switches between breathing modes often, the default mode of providing oxygen enriched air may cause the oxygen concentrator 100 to work harder, limiting the portable usage time of the system.

In an implementation, the mouthpiece 198 is used in combination with the nasal cannula airway delivery device 196 to provide oxygen enriched air to a user, as depicted in FIG. 1H. Both the mouthpiece 198 and the nasal airway delivery device 196 are coupled to an inhalation sensor. In one implementation, the mouthpiece 198 and the nasal airway delivery device 196 are coupled to the same inhalation sensor. In an alternate implementation, the mouthpiece 198 and the nasal cannula airway delivery device 196 are coupled to different inhalation sensors. In either implementation, the inhalation sensor(s) may detect the onset of inhalation from either the mouth or the nose. The oxygen concentrator 100 may be configured to provide oxygen enriched air to the delivery device (i.e. mouthpiece 198 or nasal cannula airway delivery device 196) proximate to which the onset of inhalation was detected. Alternatively, oxygen enriched air may be provided to both the mouthpiece 198 and the nasal airway delivery device 196 if onset of inhalation is detected proximate either delivery device. The use of a dual delivery system, such as depicted in FIG. 1H may be particularly useful for users when they are sleeping and may switch between nose breathing and mouth breathing without conscious effort.

Operation of oxygen concentrator 100 may be performed automatically using the internal controller 400 coupled to various components of the oxygen concentrator 100, as described herein. The controller 400 may include one or more processors 410, an internal memory 420, a transceiver 430, and a GPS receiver 434 as depicted in FIG. 1B. Methods used to operate and monitor oxygen concentrator 100 may be implemented by program instructions stored in the internal memory 420 or an external memory medium coupled to the controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Random Access Memory (RAM), etc.; or a non-volatile memory such as a magnetic medium, e.g., a hard drive, or optical storage. The memory medium may comprise other types of memory as well, or combinations thereof In addition, the memory medium may be located proximate to the controller 400 by which the programs are executed, or may be located in an external computing device that connects to the controller 400 over a network, as described below. In the latter instance, the external computing device may provide program instructions to the controller 400 for execution. The term “memory medium” may include two or more memory media that may reside in different locations, e.g., in different computing devices that are connected over a network.

In some implementations, the controller 400 includes the processor 410 that includes, for example, one or more field programmable gate arrays (FPGAs), microcontrollers, etc. included on a circuit board disposed in the oxygen concentrator 100. The processor 410 is configured to execute programming instructions stored in the memory 420. In some implementations, programming instructions may be built into the processor 410 such that a memory external to the processor 410 may not be separately accessed (i.e., the memory 420 may be internal to the processor 410).

The processor 410 may be coupled to various components of oxygen concentrator 100, including, but not limited to the compression system 200, one or more of the valves used to control fluid flow through the system (e.g., valves 122, 124, 132, 134, 152, 154, 160), the oxygen sensor 165, the pressure sensor 194, the flow rate sensor 185, temperature sensors (not shown), the fan 172, the motor speed sensor 201, and any other component that may be electrically controlled. In some implementations, a separate processor (and/or memory) may be coupled to one or more of the components.

The controller 400 is configured (e.g. programmed by program instructions) to operate oxygen concentrator 100 and is further configured to monitor the oxygen concentrator 100 for malfunction states. For example, in one implementation, the controller 400 is programmed to trigger an alarm if the system is operating and no breathing is detected by the user for a predetermined amount of time. For example, if the controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for the controller 400 to reset this alarm function. Alternatively, if the system is accidentally left on when the delivery conduit 192 is removed from the user, the alarm may serve as a reminder for the user to turn the oxygen concentrator 100 off.

The controller 400 is further coupled to the oxygen sensor 165, and may be programmed for continuous or periodic monitoring of the oxygen concentration of the oxygen enriched air passing through the expansion chamber 162. A minimum oxygen concentration threshold may be programmed into the controller 400, such that the controller 400 lights an LED visual alarm and/or an audible alarm to warn the user of the low concentration of oxygen.

The controller 400 is also coupled to the internal power supply 180 and may be configured to monitor the level of charge of the internal power supply. A minimum voltage and/or current threshold may be programmed into the controller 400, such that the controller 400 lights an LED visual alarm and/or an audible alarm to warn the user of low power condition. The alarms may be activated intermittently and at an increasing frequency as the battery approaches zero usable charge.

The controller 400 may be communicatively coupled to one or more external devices to make up a connected oxygen therapy system. The one or more external devices may be a remote external device. The one or more external devices may be an external computing device. The one or more external devices may also include sensors to collect physiological data.

FIG. 1N illustrates one implementation of a connected oxygen therapy system 450, in which the controller 400 may include the cellular wireless module 430, or other wireless communications module, configured to allow the controller 400 to communicate, using a wireless communication protocol such as the Global System for Mobile Telephony (GSM) or other protocol (e.g., WiFi), with a remote computing device 460 such as over a network. The remote computing device 460 (or a remote external device 464) such as a cloud-based server (or server 460) may exchange data with the controller 400. The remote external device may be a remote computing device, such as a portable computing device. For instance, the controller 400 may also include a short range wireless module 440 (SRWM 440) configured to enable the controller 400 to communicate, using a short range wireless communication protocol such as Bluetooth, with a portable (mobile) computing device 466 such as a smartphone. The portable computing device 466 may be associated with a user of the POC 100 in FIG. 1A. When there are two or more external devices, each external device may be the same or different. For example, when there are two external devices and each external device is the same, there may be two servers 460. Alternatively, when there are two external devices and each external device is different, there may be the server 460 and the portable computing device 466.

The server 460 may also be in wireless communication with the portable computing device 466 using a wireless communication protocol such as GSM. A processor of the smartphone/computing device 466 may execute a program known as an “app” to control the interaction of the smartphone/computing device 466 with the POC 100 and/or the server 460.

The server 460 may also be in communication with a personal computing device 464 via a wired or wireless connection to a wide-area network 470 such as the Internet or Cloud, or a local-area network such as an Ethernet. A processor of the personal computing device 464 may execute a “client” program to control the interaction of the personal computing device 464 with the server 460. One example of a client program is a browser.

Further functions that may be implemented with or by the controller 400 are described in detail in other sections of this disclosure. The controller 400 may collect data that allows the determination of a suitable dosage of oxygen supplied by the POC 100 for a patient. The controller 400 may receive physiological data from the internal sensors explained herein in the POC 100. Alternatively, the controller 400 may collect physiological or related data from the GPS receiver 434, an external blood oxygenation sensor 436, and other external sensors 438 that may be either stand-alone sensors or sensors on a health monitoring device. As will be explained further, the collected physiological data may be analyzed by the server 460 or the portable computing device 466 and additional control instructions may be provided for internal registers in the controller 400.

As will be explained, the POC 100 collects operational data and transmits the collected operational data to a remote health data analysis engine 472 that may be executed on the server 460. The health data analysis engine 472 receives the collected operational and physiological data from the POC 100 to determine a health condition of a patient based on the collected data. The health data analysis engine 472 may also determine a triggering event such as an asthma attack and determine an action to resolve the triggering event from the collected data. The health data analysis engine 472 may also receive and analyze other relevant data from other databases such as a patient information database. Other external databases may also provide additional data for the determination of the health condition. For example, a database may include “big data” from other POCs and corresponding patients. The database may also store relevant external data from other sources such as environmental data, scientific data, and demographic data. External devices such as the personal computing device 464, accessible by a health care provider, may be connected to the health data analysis engine 472, as will be explained below. Data from the database and health conditions may be further correlated by a machine-learning engine 480 as will be explained below.

Physiological and other data from additional external sensors such as the external sensor 438 in FIG. 1N may be collected. In this example, the external sensor 438 may be on a body-mounted health monitoring device. Such a device may be smart wearable clothing, smart watch, or other smart devices, in order to capture data in a low impact manner continuously from the patient. For example, the health monitoring device may include one or more sensors such as an audio sensor, a heart rate sensor, a respiratory sensor, a ECG sensor, a photoplethysmography (PPG) sensor, an infrared sensor, a photoacoustic exhaled carbon dioxide sensor, an activity sensor, a radio frequency sensor, a SONAR sensor, an optical sensor, Doppler radar motion sensors, a thermometer, or impedance, piezoelectric, photoelectric, or strain gauge type sensors. This data can be fused with other data sources collected during the day or data collected during certain periods of time, such as operational data from the POC 100. The additional external sensor 438 may also be mounted on the airway delivery device such as the nasal cannula 196. Data from the additional sensors may be sent to the mobile computing device 466 that may be in communication with the POC 100. Alternatively, data from the additional external sensors 438 on the health monitoring device may be directly sent to the POC 100. Data from the external sensors 438 on the health monitoring device, POC 100, or mobile computing device 466 may be transmitted to the network 470.

In this example, the POC 100 may include electronic components to act as a communications hub to manage data transfer with other sensors in the vicinity of the patient, and transfer of the collected data for remote processing by the health data analysis engine 472. Such data may be collected from external sensors such as the external sensor 438 on the health monitoring device by the POC 100 even when the POC 100 is not actively delivering oxygen. Alternatively, the mobile device 466 may collect data from the external sensor 438, the POC 100, and other data sources, and thus serve as a communications hub to manage data transfer to the health data analysis engine 472. Other devices such as home digital assistants that may communicate with the POC 100 may also serve as the communications hub.

An optional internal audio sensor may be embedded in the POC 100 to detect specific patient sounds. An optional external audio sensor such as a microphone may be located on the exterior of the POC 100 to collect additional audio data. Additional sensors such as a room- temperature sensor, a contact or non-contact body temperature sensor, a room humidity sensor, a proximity sensor, a gesture sensor, a touch sensor, a gas sensor, an air quality sensor, a particulate sensor, an accelerometer, a gyroscope, a tilt sensor, acoustic sensors such as passive or active SONAR, an ultrasonic sensor, a radio frequency sensor, an accelerometer, a light intensity sensor, a LIDAR sensor, an infrared sensor (passive, transmissive, or reflective), a carbon dioxide sensor, a carbon monoxide sensor, or a chemical sensor, may be connected to the controller 400 via an external port. Data from such additional sensors may also be collected by the controller 400. Data from sensors may be collected by central controller 400 on a periodic basis. Such data generally relates to the operational state of the POC 100 or its operating environment.

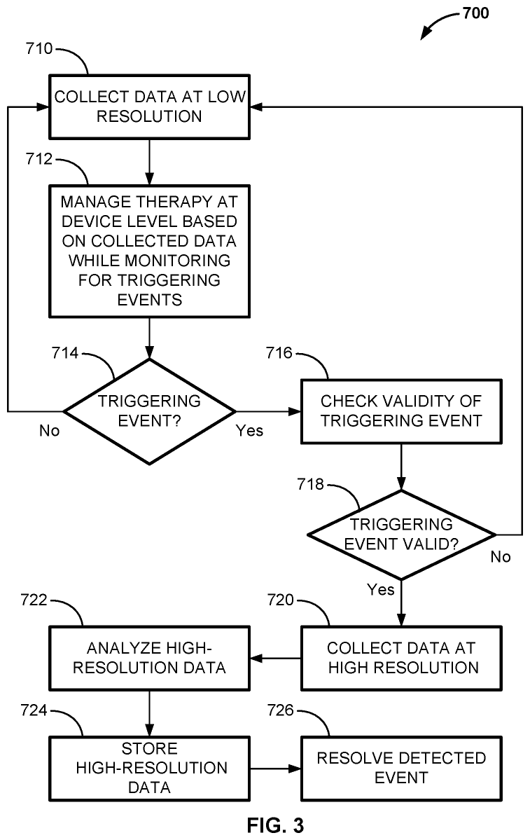

The controller 400 may collect the data at different resolutions as will be explained in greater detail in reference to a flowchart shown in FIG. 3. For example, during normal use, the data may be collected at a low resolution rate. As will be explained, a triggering event may cause the controller 400 to start collecting the data at a different resolution, such as at a higher rate and/or collecting additional types of data in a predetermined time period for more detailed analysis of the health condition of the patient. In this example, the central controller 400 encodes such data from the sensors in a proprietary data format. The data may also be coded in a standardized data format.

The control panel 600 serves as an interface between a user and the controller 400 to allow the user to initiate predetermined operation modes of the oxygen concentrator 100 and to monitor the status of the system. FIG. 1O depicts an implementation of the control panel 600. Charging input port 605, for charging the internal power supply 180, may be disposed in control panel 600.

In some implementations, the control panel 600 may include buttons to activate various operation modes for the oxygen concentrator 100. For example, the control panel 600 may include a power button 610, flow rate setting buttons 620 to 626, an active mode button 630, a sleep mode button 635, an altitude button 640, and a battery check button 650. In some implementations, one or more of the buttons may have a respective LED that may illuminate when the respective button is pressed, and may power off when the respective button is pressed again. The power button 610 may power the system on or off If the power button 610 is activated to turn the system off, the controller 400 may initiate a shutdown sequence to place the system in a shutdown state (e.g., a state in which both canisters are pressurized). The flow rate setting buttons 620, 622, 624, and 626 allow the prescribed continuous flow rate of oxygen enriched air to be selected (e.g., 0.2 LPM by the button 620, 0.4 LPM by the button 622, 0.6 LPM by the button 624, and 0.8 LPM by the button 626). In other implementations, the number of flow rate settings may be increased or decreased. After a flow rate setting is selected, oxygen concentrator 100 will then control operations to achieve production of the oxygen enriched air according to the selected flow rate setting. The altitude button 640 may be activated when a user is going to be in a location at a higher elevation than the oxygen concentrator 100 is regularly used by the user.

The battery check button 650 initiates a battery check routine in the oxygen concentrator 100 which results in a relative battery power remaining LED 655 being illuminated on the control panel 600.

A user may have a low breathing rate or depth if relatively inactive (e.g., asleep, sitting, etc.) as estimated by comparing the detected breathing rate or depth to a threshold. The user may have a high breathing rate or depth if relatively active (e.g., walking, exercising, etc.). An active/sleep mode may be estimated automatically and/or the user may manually indicate active mode or sleep mode by pressing the button 630 for active mode or the button 635 for sleep mode.

The methods of operating and monitoring the POC 100 described below may be executed by the one or more processors, such as the one or more processors 410 of the controller 400, configured by program instructions, such as including, as previously described, the one or more functions and/or associated data corresponding thereto, stored in a memory such as the memory 420 of the POC 100. Alternatively, some or all of the steps of the described methods may be similarly executed by one or more processors of an external computing device, such as the server 460, forming part of the connected oxygen therapy system 450, as described above. In this latter implementation, the processors 410 may be configured by program instructions stored in the memory 420 of the POC 100 to transmit to the external computing device the measurements and parameters necessary for the performance of those steps that are to be carried out at the external computing device.

The main use of the oxygen concentrator 100 is to provide supplemental oxygen to a user. One or more flow rate settings may be selected on a control panel 600 of the oxygen concentrator 100, which then will control operations to achieve production of the oxygen enriched air according to the selected flow rate setting. In some versions, a plurality of flow rate settings may be implemented (e.g., five flow rate settings). As described in more detail herein, the controller 400 may implement a POD (pulsed oxygen delivery) or demand mode of operation. Controller 400 may regulate the size of the one or more released pulses or boluses to achieve delivery of the oxygen enriched air according to the selected flow rate setting.

In order to maximize the effect of the delivered oxygen enriched air, the controller 400 may be programmed to synchronize the release of each bolus of the oxygen enriched air with the user's inhalations. Releasing a bolus of oxygen enriched air to the user as the user inhales may prevent wastage of oxygen by not releasing oxygen, for example, when the user is exhaling. The flow rate settings on the control panel 600 may correspond to minute volumes (bolus volume multiplied by breathing rate per minute) of delivered oxygen, e.g. 0.2 LPM, 0.4 LPM, 0.6 LPM, 0.8 LPM, 1.1 LPM.

Oxygen enriched air produced by oxygen concentrator 100 is stored in the oxygen accumulator 106 and, in a POD mode of operation, released to the user as the user inhales. The amount of oxygen enriched air provided by the oxygen concentrator 100 is controlled, in part, by the supply valve 160. In an implementation, the supply valve 160 is opened for a sufficient amount of time to provide the appropriate amount of oxygen enriched air, as estimated by the controller 400, to the user. In order to minimize the wastage of oxygen, the oxygen enriched air may be provided as a bolus soon after the onset of a user's inhalation is detected. For example, the bolus of oxygen enriched air may be provided in the first few milliseconds of a user's inhalation.

In an implementation, a sensor such as a pressure sensor 194 may be used to determine the onset of inhalation by the user. For example, the user's inhalation may be detected by using the pressure sensor 194. In use, the delivery conduit 192 for providing oxygen enriched air is coupled to the user's nose and/or mouth through the nasal airway delivery device 196 and/or the mouthpiece 198. The pressure in the delivery conduit 192 is therefore representative of the user's airway pressure and hence indicative of user respiration. At the onset of inhalation, the user begins to draw air into their body through the nose and/or mouth. As the air is drawn in, a negative pressure is generated at the end of the delivery conduit 192, due, in part, to the Venturi action of the air being drawn across the end of the delivery conduit 192. Controller 400 analyzes the pressure signal from the pressure sensor 194 to detect a drop in pressure indicating the onset of inhalation. Upon detection of the onset of inhalation, the supply valve 160 is opened to release a bolus of oxygen enriched air from the accumulator 106.

A positive change or rise in the pressure in delivery conduit 192 indicates an exhalation by the user. The controller 400 may analyze the pressure signal from the pressure sensor 194 to detect a rise in pressure indicating the onset of exhalation. In one implementation, when a positive pressure change is sensed, the supply valve 160 is closed until the next onset of inhalation is detected. Alternatively, the supply valve 160 may be closed after a predetermined interval known as the bolus duration.

By measuring the intervals between adjacent onsets of inhalation, the user's breathing rate may be estimated. By measuring the intervals between onsets of inhalation and the subsequent onsets of exhalation, the user's inspiratory time may be estimated.

In other implementations, the pressure sensor 194 may be located in a sensing conduit that is in pneumatic communication with the user's airway, but separate from the delivery conduit 192. In such implementations the pressure signal from the pressure sensor 194 is therefore also representative of the user's airway pressure.

In some implementations, the sensitivity of the pressure sensor 194 may be affected by the physical distance of the pressure sensor 194 from the user, especially if the pressure sensor 194 is located in oxygen concentrator 100 and the pressure difference is detected through delivery conduit 192 coupling the oxygen concentrator 100 to the user. In some implementations, the pressure sensor 194 may be placed in the airway delivery device 196 used to provide the oxygen enriched air to the user. A signal from the pressure sensor 194 may be provided to the controller 400 in the oxygen concentrator 100 electronically via a wire or through telemetry such as through Bluetooth™ or other wireless technology.

In some implementations, if the user's current activity level, such as that estimated using the detected user's breathing rate, exceeds a predetermined threshold, the controller 400 may implement an alarm (e.g., visual and/or audible) to warn the user that the current breathing rate is exceeding the delivery capacity of the oxygen concentrator 100. For example, the threshold may be set at 40 breaths per minute (BPM).

According to one aspect of the present technology, the controller 400 is operational to adjust the oxygen dosage to achieve a desired blood oxygenation (SpO2) level for the specific user of the POC 100. The routine executed by the controller 400 determines appropriate oxygen dosage levels to be supplied for a specific user to achieve a desired SpO2 level such as 90% or a range such as 90-95%. As explained above, a device such as the sensor 436 in FIG. 1N may be used to collect blood oxygenation data from the user of the POC 100. For example, the blood oxygenation sensor 436 may be a body-worn blood oxygenation monitor such as a wrist monitor or a waist mounted monitor. Such a monitor measures blood oxygenation by photoplethysmography. The body-worn monitor may include a transmitter that communicates with an external device such as the POC 100 (through the transceiver 430) or the portable computing device 466.

Another alternative external blood oxygenation sensor may be a finger-clip device that measures blood oxygenation levels. Such a device may also measure blood oxygenation by photoplethysmography. In this example, the finger-clip device such as the Onyx, WristOx2, or NoninConnect devices manufactured by Nonin may be in wireless communication with an external device. Another example may be an ear mounted device such as the BCI 3301 hand held pulse oximeter with 3078 ear sensor manufactured by Turner Medical. Alternatively, the user or a health care professional may take the blood oxygenation measurement and enter the measurement into an application running on the portable computing device 466.

Another alternative is that the blood oxygenation sensor 436 may be installed in the POC 100. Such a sensor may be a finger-clip device with a physical aperture in the housing of the POC 100 for a user to insert their finger. The sensor communicates the measured blood oxygenation data directly to the controller 400. Other implementations may include adapting existing body worn sensors such as a wrist-worn device such as a Fitbit, the Loop by Spry Health, the BORAband by Biosency, or an Apple watch. The external sensor 436 may be an implanted sensor that communicates data through a wireless receiver. Examples of an implanted sensor may include a skin-applied patch or a one-time nanobot implant.

In another alternative, the POC 100 itself may be a wearable device, and the blood oxygenation sensor 436 may be installed in the POC 100 so as to be in contact with the user's skin when worn. The external sensors 438 may in this implementation be mounted on the POC 100 rather than on the health monitoring device.

The SpO2 levels may also be collected at different times and locations by the POC 100 to establish different oxygen dosages for that patient to achieve the desired SpO2 level or range for different situations. Such determined “baseline” dosages may be then used to adjust the dosage of oxygen delivered by the POC 100. In one example, the user is taken through a setup procedure for the different baselines where a controller of the POC may measure the SpO2 and adjust the dosage supplied until the desired blood oxygenation level or range is achieved for that situation. The determined baselines may then be stored by the controller 400 and the controller 400 can adjust the dosage to the determined baselines when a similar situation occurs.

An example situation may be when the patient is sitting down and breathing normally. The setup procedure may be guided by instructions provided by an application executed by the portable computing device 466. For example, the application requests the SpO2 level of the patient when the patient is sitting down and breathing normally. The application records the SpO2 level and breathing rate of the user and sends the data to the controller 400. The controller 400 also sends the location of the POC 100 taken from the GPS receiver 434. After a predetermined time, such as one minute, the controller 400 adjusts the dosage of oxygen output by the POC 100 and reads a new SpO2 level. This process repeats until the desired oxygenation level or range is achieved. The controller 400 then records the final oxygen dosage as the baseline for the “sitting down and breathing normally” situation.

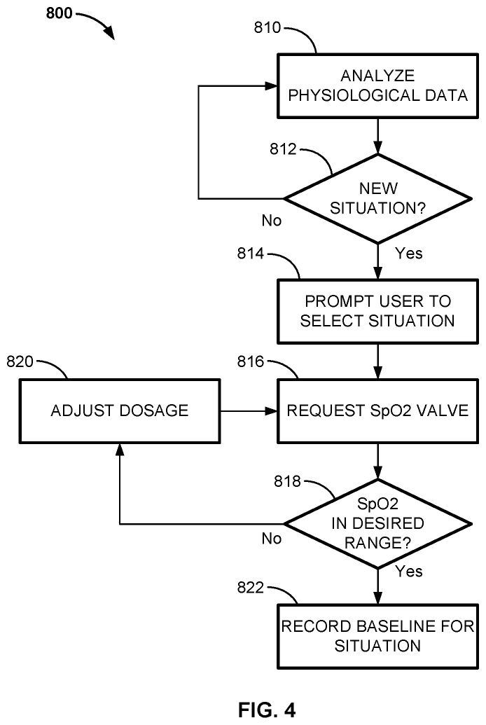

Other baselines may be determined in a similar fashion. For example, the application may detect motion based on GPS data received from the GPS receiver 434 connected to the controller 400 and determine that a user is engaging in a new activity. The application then may display an interface asking the user to enter an activity such as walking, climbing stairs, or gardening. The application then records the SpO2 level and breathing rate of the user while performing the activity and sends the data to the controller 400. After a predetermined time, such as one minute, the controller 400 adjusts the dosage of oxygen output by the POC 100 and reads a new SpO2 level. This process repeats until the desired oxygenation level or range is achieved. The baseline for the activity may then be stored by the controller 400. When the controller 400 detects that the user is performing a similar activity, the controller 400 will adjust the dosage of oxygen delivered to match the determined baseline for that activity. The other baselines may be collected over a longer period of time while a user is performing the activities. Still other baselines may be collected for different environmental conditions such as altitude or air quality. As will be explained below, FIG. 4 is a flow diagram of an example method 800 of determining a baseline dosage for a patient situation according to one implementation of the present technology.

After determination of the baselines for the user, the data may be correlated with data collected from other users to determine baseline dosage levels for a general population of users. The controller 400 stores the determined baselines and adjusts the population baselines for similar activities accordingly. The determination of the baselines may be based on data that includes SpO2 levels, blood pressure, exhaled CO₂, motion data, weight changes, etc. Other data may include patient demographic data such as weight, age, sex, race, relevant respiratory conditions/diseases, etc. In one example, machine learning such as through the health data analysis engine 472 in FIG. 1N may be used to analyze such data from the individual user as well as a general patient population to refine the correlation between oxygen dosage and SpO2 levels. The example machine learning algorithm may be executed by the controller 400 or on a remote device such as the server 460.

The machine learning algorithm may determine different general population baselines for different subsets of the general population. For example, baselines may be established for a subset of the general population with similar demographic traits such as weight, age, sex, race, relevant respiratory conditions/diseases, etc. Other baselines may be established for a subset of the general population that encounter similar environmental conditions such as living in locations at higher altitudes, being in areas of different air quality, etc. Still other baselines may be established for a subset of the general population that participate in similar physiological activities such as running, climbing, etc.

The controller 400 may also perform closed-loop therapy based on continuous monitoring of SpO2 levels. In a closed-loop therapy implementation, the blood oxygenation level is collected from the sensor 436. The controller 400 then adjusts the dosage of oxygen to achieve the desired oxygenation level or range. For example, if the SpO2 level is not sufficiently high, the controller 400 will increase the dosage of oxygen supplied. If the SpO2 level is too high, the controller 400 will decrease the dosage of oxygen supplied. When the SpO2 level is determined to be at a stable level within the desired range for a sufficient amount of time, the dosage is set by the controller 400.

The closed-loop algorithm may also identify a “dead dosage” level above which no further increase in SpO2 level may be achieved. Thus, the dead dosage level sets the limit of oxygen that will be supplied by the controller 400 to the user. Additional data may be collected to determine the correlation between the dosage and the increase in SpO2 level. Such data may include environmental factors that may affect the SpO2 level of the user. For example, location information from the GPS receiver 434 may be used to determine the current altitude. Alternatively, the altitude may be determined by from an altimeter sensor. The closed-loop algorithm may determine the dosage of oxygen based partially on the altitude sensed by the altitude sensor.

Another environmental factor may be current air quality. Air quality data may be obtained from an external database. Alternatively, the current air quality in the vicinity of the POC 100 may be determined from the output of an air quality sensor that is located in the home of the patient and is in communication with the controller 400. Big data may be collected to provide the correlation between oxygen and the SpO2 level increase to become an industry standard.

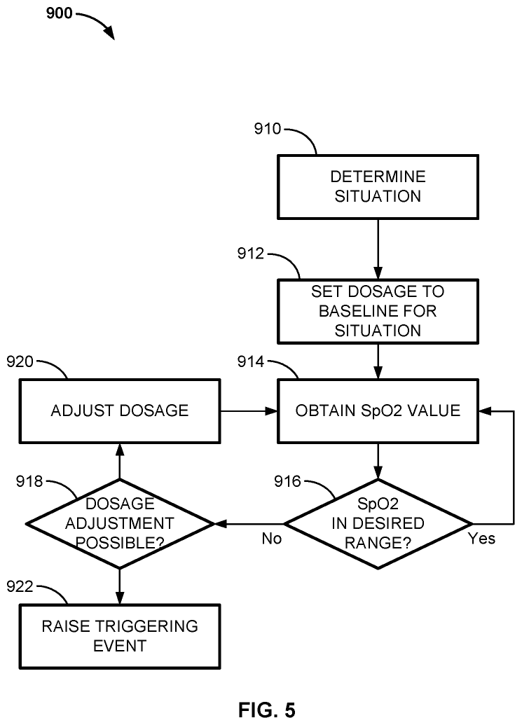

According to one aspect of the present technology, the management of the health condition of the patient by connected oxygen therapy system 450 may take place on two levels: the device level, and the cloud level. The device-level management involves repeated adjustments by the controller 400 to the oxygen dosage based on the collected sensor data. The closed-loop therapy method 900 described below in relation to FIG. 5 is one example of device-level management. However, the connected oxygen therapy system 450 may transition to cloud-level management upon detection of a triggering event. One example of a triggering event is the triggering event, indicative of the inability of the POC 100 to adjust the oxygen dosage sufficiently to maintain the patient's blood oxygenation in the desired range. Cloud-level management may include temporarily collecting data at a higher resolution, determination of the health condition of the patient based on the collected data, and recommending or carrying out an action to resolve the triggering event. The cloud-level management may be coordinated by a remote computing device such as the server 460.

According to one aspect of the two-level management scheme, the POC 100 may change the resolution of data that is collected and uploaded to the network 470 based on a triggering event and/or for a specific purpose that requires greater detail in the collected data. These smart health insights into co-morbidities with high resolution data and sensing can be enabled by changing from low resolution data collection to high resolution data collection, augmented by cloud processing from the health data analysis engine 472.

In this aspect, the POC 100 may collect operational and physiological data at a relatively low sampling rate, such as 0.1 Hz (6 samples per minute). Optional external sensors that may be connected to the POC 100 may allow other data as explained above to be collected. In addition, standard annotations for exchange and storage of medical data, such as the European Data Format (EDF), may be applied to the collected data. As will be explained, the above data may be collected at a higher sampling rate. In addition, the high resolution mode may allow the collection of additional types of data or data derived from the basic data.

In the connected oxygen therapy system 450, the example POC 100 serves as a connected hub in the home environment. The hub role is combined with the external health data analysis engine 472 for managing health conditions such as COPD, asthma, emphysema, and chronic bronchitis in this example. This could be offered as care as a service to an integrated payor. The POC 100 and associated external sensors 438, such as those mounted on the health monitoring device, can monitor all aspects of disease that the patient may be suffering from. For example, the analysis engine 472 may predict exacerbations (e.g., Asthma attack, COPD exacerbation) before they occur, based on triggering events that may be determined from the collected data. Other diseases and health conditions may be monitored based on the correlation of the collected data. Such correlations may be determined by the machine-learning engine 480 in FIG. 1N.

In one implementation, there are different triggering events that may result in an increase in the resolution of data collection from the POC 100. A low resolution collection mode may include collection of standard operational data such as compressor motor speed or dosage of oxygen delivered. A high resolution collection mode may increase the collection rate and/or types of data that may be used for analyzing health conditions over a predetermined period of time in comparison to the low resolution collection mode. The process of changing to the high resolution collection mode is based on the triggering event determined from data collected at the low rate collection mode. A determination is made as to whether a triggering event occurs, and what the event signifies.

After a triggering event is detected, there can be a determination of whether the triggering event was real. Verification of a real triggering event can be based on data from multiple sensors or duration of the event. If the determination is made that the triggering event was not real, the collection of data reverts back to the resolution of data collection before the triggering event. Optionally, the devices involved in the triggering event can perform diagnostics to determine if there is an issue/error that generated the false triggering event. If the triggering event reflects a real event, the processing can proceed based on data collection in a higher resolution mode.

The data resolution that changes can be a higher data rate that is sampled by the device and uploaded to the cloud or a higher data rate that is uploaded to the cloud but already sampled by the device. The sampling may be made from the sensors such as those shown in FIG. 1N. Additional sensors can startup/shutdown to control (increase/decrease) the amount of data and/or data rate.

In the event of a real triggering event, a severity check can occur to determine the urgency or severity required of responsive action. The severity determination is based on how severe the triggering event may be. For example, a low severity event may trigger a notice to the patient to take action. A high severity event may require action taken by clinician or a call for emergency response. The determination of the triggering event and the evaluation of the severity of the triggering event may be based on rules or controlled automatically by the machine-learning engine 480. The machine-learning engine 480 may be taught to detect different triggering events based on a training set of collected input data and triggering events. Similarly, the machine-learning engine 480 may be taught to determine the severity of different triggering events and the appropriate response. The weights to determine both a triggering event and the level of response may be refined by the machine-learning engine 480 as additional data is collected, and an evaluation of the resulting outcomes is made.

For example, one triggering event may be a request to build up health records for the user or patient that may be initiated by the patient or a caregiver. Such a request may be provided remotely via the mobile device 466 or a remote external device such as the workstation 464 in FIG. 1N. For example, the request to build up a health record may be in order to establish a new baseline for the user such as that described above in relation to blood oxygen saturation, to detect normal trends of the user to compare to longitudinal data in the future, or to access electronic health records. Alternatively, the baseline may be determined by adding the data from the patient to a baseline relating to a patient population of normative values for the type of patient.

The collected physiological data for such requests may include blood oxygenation values, averaged (such as running mean or median) blood oxygenation, a baseline blood oxygenation, trends in blood oxygenation over different time scales, or blood oxygenation variability over different time scales. Different timescales could be a second, minute, hour, multiple hours, a day, or other intervals. Blood oxygenation values may also be related to day or night times, whether the user is asleep, and in what sleep stage, whether the user is sitting quietly, exercising, and any other relevant factors. The collected data could include movement metrics that are processed to estimate activity over different timescales and processing. This data could be processed to calculate energy expenditure of the patient.

Other examples of physiological data is estimates of dyspnea generated based on breathing rate, or feelings of difficulty breathing or being smothered that may be reported by the patient through an application running on the mobile device 466.

Operational parameters of the POC 100, such as oxygen dosage and compressor speed, can be tracked over time as will be explained below. Any of these parameters may be compared to global or personalized thresholds to generate triggering events.

Another example of a triggering event is an unusual hiatus in a patient using a POC. The interruption of low resolution data collection from the POC may alert a health care provider or patient that the patient is away from home and has not brought the POC with them. Another interruption scenario may be that the patient is too sick to operate the POC. In such a case, other environmental sensors such as a smart assistant, mattress or bed sensor, light sensor, temperature sensor, occupancy sensor, passive infrared (PIR) sensor, video/security camera, radio frequency (RF) imaging sensor (such as UWB or IR-UWB), microwave sensor, building or home smart management system, intruder alarm sensors and setting (current mode such as enabled or disabled), smart meter (measuring electricity usage), door operated switch, audio sensor, fall detection sensor, presence of a smartphone and/or Bluetooth beacon, smartphone battery/charge/usage including user interface (UI) interactions or movements, may be accessed to check the condition of the patient.

Another example triggering event may be if a consumable, such as a sieve bed or a battery, has been depleted, preventing use of the POC 100. The system may then provide communication to a supply system to drop-ship a replacement consumable directly to the patient, or provide delivery of the consumable to a pharmacy for patient pickup or to a technician for installation at the patient location.

Another example of a triggering event for higher resolution data collection is the provision of new medication or treatment. For example, if new medication is provided (either new to the user or new to the market) where the user is embarking on a new or changed drug regime, the connected oxygen therapy system can monitor physiological parameters at a higher collection resolution (and potentially carry out gas analysis) to determine the results of the new treatment and check that it does not have side effects or interactions with other medications. Key data may include changes in breathing rate, changes in activity level, reduction in patient-reported discomfort, increase in patient-reported energy levels, reduction in selected oxygen dosage, increase in usage of the POC, and reduction in patient-reported depression or anxiety.

A similar procedure may be used to evaluate medication or other treatment for the patient. The medication amount or frequency of dosing may be adjusted automatically if the patient receives medication via a platform integrated with the POC 100. Such a delivery platform may include a drug reservoir. For example, the drug reservoir may be used in conjunction with a patch for daytime delivery (thus saving power and medication contained in the patch). The POC 100 may thus control the drug reservoir to administer the medication while the POC 100 is in use. Thus, a platform for medication delivery is realized with routine delivery of medication or exceptional delivery of medication based on a triggering event. Based on clinical review and approval, certain medications such as bronchodilators, anti-inflammatories, and antibiotics may be delivered to the patient. These may be delivered while the patient is awake and wearing an airway delivery device. Delivery of such medications may be made by pressing a button. This delivery may be combined with modified oxygen dosages from the POC 100 or the addition of other therapy devices, for example.

The mobile device 466 may include an application that communicates additional instructions to the patient. For example, an application may also ask the patient to calm down and may contact emergency services or another health care provider. Similarly, medication may be offered for patients having (or predicted to have) COPD exacerbations. Other medications, such as nasal decongestants, may treat an upper airway infection related to the common cold or influenza (e.g., to try to prevent this deteriorating into an exacerbation). Other devices that administer medication may be communicatively integrated with the POC 100. For example, medication may be delivered via a smart inhaler, a combination monitoring accessory attached to an inhaler such as those offered by Propeller Health, or other network-connected metered medication delivery system/devices, that may communicate the occurrence of dosages of medication inhaled by the patient and other data.

The collected data may be used to determine respiration changes for the tracking of changes in conditions, such as COPD (e.g., higher than normal breathing rate/tachypnea). The collection of respiration data over time may determine how base (e.g., an average “baseline” level) respiration rate evolves over time. Such disease analysis may include tracking worsening disease conditions. For example, the collected data may be analyzed to detect worsening Asthma, pollen allergy, common cold, or respiratory tract infections.

Audio data may be used to confirm or enhance health data analysis. For example, sounds of breathing from a patient from an internal audio sensor may be used in conjunction with external sounds detected by an external audio sensor to determine audio data.

The audio data may include the level of residual snoring, gasping, wheezing, spluttering, and the sound of the heartbeat. These sounds may be used to determine respiratory and other health conditions. For example, the intensity and timing (inspiration or expiration) of a wheeze sound may be a symptom of respiratory conditions, disorders, or ailments. In addition, lack of sounds, such as a silent chest, may indicate severe asthma in combination with other vital signs like a higher heart rate and respiration rate.

The collected operational and physiological data may be analyzed in the context of patient-specific conditions that may be derived from data from other sources. Such data may include outcomes reported by the patient through an application running on the mobile device 466, or input from electronic health records on a database. The patient-specific data may therefore include conditions a patient may have such as pre-existing issues, demographic details (BMI, age, gender), and geographic details (allergen risks due to pollen count, heat exhaustion due to outside temperature, air quality and oxygen quantity due to altitude), and medications associated with the patient.

The patient-reported outcomes may include subjective feedback on how the patient is feeling, whether the patient feels fatigued, and the level of sleepiness. The data may be used to determine the quality of sleep for the patient. This may be a comparison to a personal baseline for the patient, linked to weather data, a comparison to an average sleeper of their age and gender (aiming to be better than average), or a comparison to an average of a person with the same chronic conditions and or disease progression. As explained above, the baseline may be determined from a normative patient population relative to the patient. Such data may be displayed in an application executed by the mobile device 466. Such data may also be made available on a connected workstation such as the workstation 464 for a health care professional.

A sensor on the POC 100 may sense gas (breath) from the patient using for example an array of cross-reactive sensors, and pattern recognition/deep learning to identify the characteristic changes in certain volatile organic compounds (VOC)s due to disease progression. Additional sensors may detect gases, fumes, smoke, or particulates, in a room environment where the POC 100 is located as well in the exhaled gas of the patient (such as measuring the quantity of PM2.5 (inhalable particles with a diameter of generally 2.5 micrometers and smaller) and PM10 (particles with a diameter of 10 micrometers and smaller). Such data may be used to determine whether bacteria, viruses, and other contaminants have been adequately filtered, such as via a HEPA filter removing 0.3 micron particles and smaller, which includes most mold, mildew, and viruses.

As explained above, another device such as a smartphone, smart device, smart speakers such as Alexa or Google Home, or smart services such as Siri or Bixby may include an application that may trigger the higher resolution collection of data. For example, audio sensing may run on the device to listen for breath sounds, such as inspiration, expiration, coughing, wheezing, snuffling, sneezing, gasping, and whistle. Acoustic sensing may also be active using SONAR to sense a reflected signal from the chest. An RF sensor may be integrated into a smart device to detect ballistocardiogram (movement or heart), movement of the chest and abdomen with breathing, or activity.

A data collection resolution increase may be triggered if a new sensor or other data streams become available. This could include allocating more processing resources to analyze more detailed waveforms (such as requiring a higher rate of data transfer, and potentially higher energy consumption) for a period of time to identify and manage a new or worsening condition. In addition, a new device (such as a new POC) may trigger the increased rate of collection of data. The higher resolution data may allow a change in device, calibration to new settings, and configuration with an optimized profile of the user. The higher resolution data may be used to confirm that the POC 100 is functioning as expected.

An example triggering event may be ECG data derived from the collected data showing signs of left ventricular hypertrophy, presence of atrial fibrillation (irregularly irregular beats), or tachycardia. The connected oxygen therapy system may then determine the events signify a possible stroke. Such data may also signify a heart attack, stress (heart rate averages do not reduce as expected during sleep), or COPD exacerbation.

Such triggering events may also indicate incorrect POC 100 device settings, an uncompliant user, blood pressure, asthma attack, and arrhythmias such as diabetes. Generally, a responsive action is then determined by the analysis. The patient may be notified, and the patient may take a recommended action. For example, the actions may include actions that may be communicated to the patient via the mobile device 466. For example, a message may be sent to the patient to check a bio-signal related to the event. The actions may also include a notification that indicates that medication is required, which the patient may already have in their possession. The event may be to prompt the patient to report a current feeling or health condition. For example, the application on the mobile device 466 may ask a patient to input responses to inquiries such as “how do you feel” or other questions in relation to the current state of the patient. Such data may be collected from patient input to the application in the form of a slider or a numerical rating. Such subjective information may provide additional verification to cross-check collected objective data streams. The responses of the patient may therefore be used as a feedback mechanism to reduce risk of a false positive triggering event. The responses may also be compared to previous patient responses. A change in one or some of these user-reported outcomes (and increase in perceived quality of life such as increased alertness, ability to walk, or walk a longer distance, not feeling breathless, or clearing up a respiratory infection), along with an improvement in the collected data may be captured by the system. An application on the mobile device 466 may also suggest that the patient consult a health care professional. The application may give the patient the underlying data generated from the health data analysis engine 472. The application may provide the data to indicate good health, which can be used for rewards or incentives such as a health insurance discount. The patient may be provided coaching based on the granular data.

In other examples, other actors such as payors may take action in response to a triggering event. For example, the payor could be a government program, employer, insurance provider, or doctor. Information may allow the payor to request the patient come to a health care facility for new tests, as the information may indicate a new and/or unknown/undiagnosed condition. The payor may determine the onset of respiratory conditions or disease and avoid re-admission of the patient. Clinician-specific analysis may allow a health care provider to focus on which patients to insure/treat, and track the efficacy of medications/treatments.

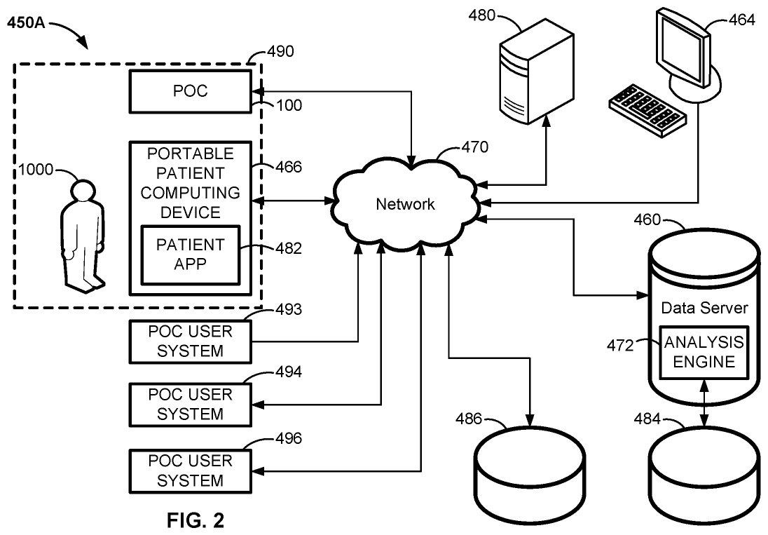

FIG. 2 illustrates another implementation 450A of a connected health care data collection and analysis system, in which the controller 400 of the POC 100 includes the transceiver 430 as in FIG. 1N configured to allow the controller 400 to communicate, using a wireless communication protocol such as the Global System for Mobile Telephony (GSM) or other protocol (e.g., WiFi), with a remote external device (or remote computing device) such as the cloud-based server 460 over the network 470. The network 470 may be a wide-area network such as the Internet or cloud, or a local-area network such as an Ethernet. Alternatively, or in addition, the remote external device may be a remote computing device, such as the portable computing device 466. For instance, the controller 400 may also include the short range wireless module 440 configured to enable the controller 400 to communicate, using a short range wireless communication protocol such as Bluetooth™, with the portable computing device 466 such as a smartphone. The smartphone portable computing device 466 may be associated with a user 1000 of the POC 100.

The server 460 may also be in wireless communication with the portable computing device 466 using a wireless communication protocol such as GSM. A processor of the smartphone 466 may execute an application 482 to control the interaction of the smartphone with the POC 100 and/or the server 460. In this example, the application 482 may collect data for determining the oxygen dosage of the POC 100. The application 482 may also include input interfaces for the user 1000 to input data such as SpO2 readings from external sensors if a blood oxygenation sensor such as the sensor 436 in FIG. 1N are not available.

The server 460 includes an analysis engine 472 that may execute operations such as a baseline determination algorithm for different users or a machine learning algorithm with different types of input data to determine the correlation between SpO2 levels and oxygen dosages supplied by the POC 100. The server 460 may also be in communication with other devices such as a personal computing device workstation 464 via a wired or wireless connection via the network 470. The server 460 has access to a database 484 that stores operational data about the POCs and users managed by the system 450. The database 484 may be segmented into individual databases such as a user database having information about users of the POCs and operational data associated with the POC use. The server 460 may also be in communication via the network 470 with other relevant databases such as an environmental database 486 that may provide additional data.

The user 1000 of the POC 100 and portable computing device 466 may be organized as a POC user system 490. The connected system 450A may comprise multiple POC user systems 490, 492, 494 and 496 that each include a POC user, POCs such as the POC 100, and portable computing devices such as the portable computing device 466. Each of the other POC user systems 492, 494 and 496 are in communication with the server 460, either directly or via respective portable computing devices associated with respective users of the POCs. Controllers corresponding to the controller 400 and transceivers corresponding to the transceiver 430 in each of the POCs of the systems 492, 494 and 496 collect the data described above in relation to FIG. 1N. The workstation such as the personal computing device 464 may be associated with a health management entity (HME) that is responsible for the therapy of a population of users of the fleet of POCs.

Data from the databases 484, health conditions from the health data analysis engine 472 and data from individual POC user systems such as the user system 490 may be further correlated by the machine-learning engine 480. The machine-learning engine 480 may implement machine-learning structures such as a neural network, decision tree ensemble, support vector machine, Bayesian network, or gradient boosting machine. Such structures can be configured to implement either linear or non-linear predictive models for determining different health conditions. For example, data processing such as classification of a health condition may be carried out by any one or more of supervised machine learning, deep learning, a convolutional neural network, and a recurrent neural network. In addition to descriptive and predictive supervised machine learning with hand-crafted features, it is possible to implement deep learning on the machine-learning engine 480. This typically relies on a larger amount of scored (labeled) data (such as many hundreds of data points from different POC devices) for normal and abnormal conditions. This approach may implement many interconnected layers of neurons to form a neural network (“deeper” than a simple neural network), such that more and more complex features are “learned” by each layer. Machine learning can use many more variables than hand-crafted features or simple decision trees.

Convolutional neural networks (CNNs) are used widely in audio and image processing for inferring information (such as for face recognition), and can also be applied to audio spectrograms, or even population scale genomic data sets created from the collected data represented as images. When carrying out image or spectrogram processing, the system cognitively “learns” temporal and frequency properties from intensity, spectral, and statistical estimates of the digitized image or spectrogram data.

In contrast to CNNs, not all problems can be represented with fixed-length inputs and outputs. For example, processing respiratory sounds or sounds of the heart has similarities with speech recognition and time series prediction. Thus, the sound analysis can benefit from a system to store and use context information such as recurrent neural networks (RNNs) that can take the previous output or hidden states as inputs. In other words, they may be multilayered neural networks that can store information in context nodes. RNNs allow for processing of variable length inputs and outputs by maintaining state information across time steps, and may include LSTMs (long short term memories, types of “neurons” to enable RNNs increased control over, which can be unidirectional or bidirectional) to manage the vanishing gradient problem and/or by using gradient clipping.

The machine-learning engine 480 may be trained for supervised learning of known health conditions from known data inputs for assistance in analyzing input data. The machine-learning engine 480 may also be trained for unsupervised learning to determine unknown correlations between input data and health conditions, to increase the range of analysis of the health data analysis engine 472.

The collection of data from the population of users of the fleet of POC user systems such as user systems 492, 494 and 496 allows large population health data to be collected for the purpose of establishing baselines and providing more accurate health data analysis. As explained above, the collected data may be supplied to the machine-learning engine 480 for further analysis of correlations to determine health conditions and predict exacerbations. The analysis from either the health data analysis engine 472 and/or the machine-learning engine 480 may be used to provide health data analysis for any of the individual patients such as the patient 1000.

FIG. 3 shows a flow diagram for a data collection and analysis routine 700 performed by the system 450 in FIG. 1N or 450A in FIG. 2. FIG. 4 is a flow diagram of a method 800 of determining a baseline dosage for a patient situation according to one implementation of the present technology. FIG. 5 is a flow diagram of a method 900 of performing closed-loop therapy based on continuous monitoring of SpO2 levels according to one implementation of the present technology. The flow diagrams in FIGS. 3-5 are representative of example routines implementable by machine-readable instructions for the health data analysis engine 472 or the controller 400 to collect and analyze data for determining health conditions for each patient such as the patient 1000 in FIG. 2. In this example, the machine-readable instructions comprise an algorithm for execution by (a) a processor; (b) a controller; and/or (c) one or more other suitable processing device(s). The algorithm may be embodied in software stored on tangible media such as flash memory, CD-ROM, floppy disk, hard drive, solid-state drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application-specific integrated circuit (ASIC), a programmable logic device (PLD), a field-programmable logic device (FPLD), a field-programmable gate array (FPGA), discrete logic, etc.). For example, any or all of the components of the interfaces can be implemented by software, hardware, and/or firmware. Also, some or all of the machine-readable instructions represented by the flowcharts may be implemented manually. Further, although the example algorithm is described with reference to the flowcharts illustrated in FIGS. 3-5, persons of ordinary skill in the art will readily appreciate that many other methods of implementing the example machine-readable instructions may alternatively be used. For example, the order of execution of the blocks may be changed, and/or some of the blocks described may be changed, eliminated, or combined.

As shown in FIG. 3, the system first monitors data collected in a low resolution mode from the POC 100 (710). In this example, the data analysis engine 472 collects the low resolution data and analyzes the data for a triggering event, such as a change in the condition of the patient. As explained above, the triggering events may be detected from predetermined rules or machine-learning-based models or algorithms. The analysis engine 472 determines whether a triggering event has occurred based on the collected data from the low resolution mode (712). If there is no triggering event detected (“NO” at 714), the routine continues to collect data at a low resolution (710). While the analysis engine 472 is monitoring the low resolution data (712), the therapy is managed at the device level by the controller 400 of the POC 100, for example in accordance with the method 500 described below.

If a triggering event has been detected (“YES” at 714), the health data analysis engine 472 confirms the triggering event (716). The confirmation of the triggering event may include collecting additional types of data such as patient-reported outcomes or following a set rule to cross-check the occurrence of the triggering event. Such collection may be made on an application on the mobile device 466. The analysis engine 472 then determines whether the triggering event is confirmed (718). If the triggering event is not confirmed, the routine continues to collect data at a low resolution (710). If the triggering event is confirmed, the analysis engine 472 controls the POC 100 and any other relevant sources to increase the collection of data to a high resolution for a predetermined period (720). As explained above, increasing the resolution may involve collecting additional data from the external sensors 438 mounted on the patient health monitoring device, other sensors, and other databases. The data analysis engine 472 then analyzes the collected data to determine a health condition (722). The health data analysis may include determining the severity of the triggering event. The collected analysis is then stored (724) for further analysis and other purposes such as building on a training set for the machine-learning engine 480. The health data analysis engine 472 then determines and implements a response to the detected triggering event (726), possibly based on the determined severity. For example, this may involve notifications to the patient in less severe triggering events or notifications to a health care professional in more severe triggering events. Other examples of responses may be adjustment of the settings of the POC 100 and automatic adjustment of other treatment, e.g. medication. Once the data analysis engine 472 determines the triggering event has been resolved, it returns to the collection of data at a low resolution (710).

FIG. 4 shows the flow diagram of the routine 800 run by either the health data analysis engine 472 or the controller 400 to determine a baseline dosage of oxygen supplied by the POC 100. The routine 800 first analyzes the physiological data collected by sensors such as the sensor 436 or sensor 438 in FIG. 1N (810). The routine then determines whether the collected physiological data indicates a new health situation (812). If no new situation is determined, the routine cycles back to collect additional physiological data (810). If a new situation is determined, the routine prompts the user to select the situation indicated by the physiological data (814). The routine then requests the SpO2 value from the POC 100 (816). The routine determines whether the SpO2 value is in the desired range (818). If the SpO2 is not in the desired range, the routine adjusts the oxygen dosage of the POC 100 (820). The routine then cycles back and reads the new SpO2 value (816). If the SpO2 value is in the desired range, the routine records the baseline for the situation in memory (822). The POC 100 may then supply the baseline dosage when a similar situation is detected from the physiological data.

FIG. 5 shows the flow diagram of by the routine 900 run by either the health data analysis engine 472 or the controller 400 to adjust the dosage of oxygen supplied by the POC 100. Physiological data and POC operational data are collected to determine the situation of the patient (910). The oxygen dosage supplied to the patient is set to the baseline dosage for the determined situation (912). As explained above in relation to the routine 800 in FIG. 4, the controller 400 or health data analysis engine 472 may determine appropriate baselines for different situations for a patient. The routine then requests the SpO2 value from the POC 100 (914). The application determines whether the SpO2 value is in the desired range (916). If the SpO2 value is in the desired range, the routine continues to obtain the SpO2 value (914). If the SpO2 is not in the desired range (916), the routine determines whether a dosage adjustment is possible within the capacity of the POC 100 (918). If an adjustment is possible, the routine adjusts the oxygen dosage of the POC 100 to attempt to get the SpO2 value within the desired range (920). The routine then cycles back and reads the new SpO2 value (914). If the oxygen dosage cannot be adjusted, the routine raises a triggering event (922).

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A system for managing a respiratory condition of a patient, the system comprising:
an oxygen concentrator configured to:
generate and deliver oxygen enriched air to the patient according to a selected dosage;
sense and collect physiological data of the patient;
collect operational data during the generation and delivery of oxygen enriched air;
adjust the dosage of oxygen enriched air based on the sensed physiological data; and
transmit operational data and the physiological data; and
a health data analysis engine in communication with the oxygen concentrator, the health data analysis engine being configured to:
collect the data transmitted by the oxygen concentrator;
detect a triggering event based on the collected data; and
determine a responsive action to resolve the detected triggering event.

2. The system of claim 1, wherein the oxygen concentrator is a portable oxygen concentrator, and wherein the oxygen concentrator includes an air intake, a motorized compressor coupled to the air intake, an oxygen separator to separate oxygen from compressed air from the compressor, and an accumulator coupled to the oxygen separator to store the oxygen enriched air.

3. The system of claim 1, wherein the oxygen concentrator has a first data resolution and a second data resolution, the second data resolution including comparatively more data than the first data resolution, wherein the operational and physiological data is collected at the second data resolution during a predetermined period of time when the triggering event is detected.

4-6. (canceled)

7. The system of claim 1, wherein the operation of the oxygen concentrator is adjusted in response to the triggering event.

8. (canceled)

9. The system of claim 1, further comprising a health monitoring device coupled to the oxygen concentrator, the health monitoring device comprising one or more sensors configured to collect the physiological data, wherein the one or more sensors of the health monitoring device are selected from the group consisting of: an audio sensor, a heart rate sensor, a respiratory sensor, a ECG sensor, a photoplethysmography (PPG) sensor, an infrared sensor, a photoacoustic exhaled carbon dioxide sensor, an activity sensor, a radio frequency sensor, a SONAR sensor, an optical sensor, a Doppler radar motion sensor, a thermometer, an impedance sensor, a piezoelectric sensor, a photoelectric sensor, and a strain gauge sensor.

10. (canceled)

11. The system of claim 1, wherein the health data analysis engine is operable to predict respiratory exacerbations based on the operational data and the physiological data.

12-14. (canceled)

15. The system of claim 1, wherein the responsive action is one of the group of: an adjustment to a treatment of the patient; a notification to the patient to adjust a treatment; or a notification to a healthcare professional to adjust a treatment.

16-19. (canceled)

20. The system of claim 1, wherein the dosage of oxygen enriched air is determined by comparison to a baseline determined from previous physiological data from the patient.

21-23. (canceled)

24. The system of claim 1, further comprising a blood oxygenation sensor coupled to the oxygen concentrator, wherein the physiological data includes blood oxygenation data of the patient measured by the blood oxygenation sensor.

25. The system of claim 24, wherein a dead dosage level is identified for the patient where no further increase in blood oxygenation may be achieved.

26. A method of managing a respiratory condition of a patient, comprising:
generating oxygen enriched air via an oxygen concentrator delivering the oxygen enriched air to the patient according to a selected dosage;
sensing physiological data of the patient;
collecting operational data during the generation and delivery of oxygen enriched air from the oxygen concentrator;
adjusting the dosage of oxygen enriched air based on the sensed physiological data;
transmitting the operational data and the physiological data to a health data analysis engine in communication with the oxygen concentrator;
collecting the data transmitted by the oxygen concentrator;
detecting a triggering event based on the collected data; and
determining a responsive action to resolve the detected triggering event.

27. The method of claim 26, wherein the oxygen concentrator is a portable oxygen concentrator, and wherein the oxygen concentrator includes an air intake, a motorized compressor coupled to the air intake, an oxygen separator to separate oxygen from compressed air from the compressor, and an accumulator coupled to the oxygen separator to store the oxygen enriched air.

28. The method of claim 26, wherein the oxygen concentrator has a first data resolution and a second data resolution, the second data resolution including comparatively more data than the first data resolution, wherein the operational and physiological data is collected at the second data resolution during a predetermined period of time when the triggering event is detected.

29-31. (canceled)

32. The method of claim 26, further comprising adjusting the operation of the oxygen concentrator in response to the triggering event.

33. (canceled)

34. The method of claim 26, further comprising collecting physiological data via a health monitoring device coupled to the oxygen concentrator, wherein the health monitoring device comprises one or more sensors configured to collect the physiological data, wherein the one or more sensors of the health monitoring device are selected from the group consisting of: an audio sensor, a heart rate sensor, a respiratory sensor, a ECG sensor, a photoplethysmography (PPG) sensor, an infrared sensor, a photoacoustic exhaled carbon dioxide sensor, an activity sensor, a radio frequency sensor, a SONAR sensor, an optical sensor, a Doppler radar motion sensor, a thermometer, an impedance sensor, a piezoelectric sensor, a photoelectric sensor, and a strain gauge sensor.

35. (canceled)

36. The method of claim 26, further comprising predicting respiratory exacerbations based on the operational data and the physiological data.

37-44. (canceled)

45. The method of claim 26, wherein the dosage of oxygen enriched air is determined by comparison to a baseline determined from previous physiological data from the patient.

46-48. (canceled)

49. The method of claim 26, wherein the physiological data includes blood oxygenation data of the patient measured by a blood oxygenation sensor.

50-51. (canceled)

52. A non-transitory computer readable medium having stored thereon software instructions that, when executed by a processor, cause the processor to:
sense physiological data of the patient;
collect operational data during the generation and delivery of oxygen enriched air from an oxygen concentrator generating oxygen enriched air and delivering the oxygen enriched air to a patient according to a selected dosage;
adjust the dosage of oxygen enriched air based on the sensed physiological data;
transmit the operational data and physiological data of the patient;
detect a triggering event based on the collected data; and
determine a responsive action to resolve the detected triggering event.
